SEQUENCE LISTING (5 sequences)

SEQ ID NO: 1 - DNA, 440 bp, Beet yellows virus
ttaatgatggtgaaacttctcgagccatatctaggtctgaatcgctcctacgtcgtgtgaaggaacttggaacgaattcgccgcaaagcgaagtctctgagtgcatcaacgaattcaacgaactagcaagatttaatcacttgttagttacggtcgaacacagggaatggatggaaaaacatccgaaacagtcgtcggaacttcgagctccgtcacgacttggcgaaatgctcaaagaaattcgtgcgttccttaaggtgcgcgtggtaactcccatgcataaggagactgccagcgaaactctgaacgcgtttctggaagagtactgccgaattgctggactcactcgcgaggatgccttgcgagaaaaaatgaggaaagtaaggagtacggtgctgtttcatcactcggaacttttgaagttcgaagtgactgaaaac

SEQ ID NO: 2 - DNA, 450 bp, Beet mild yellowing virus
tctcagccagtggttgtggtccaaacctctcggacaacacaacgccgacctagacgacgacgaagaggtaacaaccggacaggaagaactgttcctaccagaggagcaggttcgagcgagacatttgttttctcaaaagacaatctcgcgggaagttccagcggagcaatcacgttcgggccgagtctatcagactgcccggcattcgctaatggaatgctcaaggcctaccatgagtataaaatctcaatggtcattttggagttcgtctccgaagcctcttcccaaaattccggttccatcgcttacgagctggacccacactgtaaactcaactccctttcctcaactatcaacaaattcgggatcacaaagcccgggaggagggcgtttacagcgtcttacatcaacgggacagaatggcacgacgttgccaaggaccaattcagg

SEQ ID NO: 3 - DNA, 444 bp, Beet curly top virus
atccggcaacctcatctccacgtcctgcttcaactcgaaggaaaagttcagatcacaaatatcagattattcgacctggtatccccaaccaggtcagcacatttccatccaaacattcagagagctaaatccagctccgacgtcaagtcctacgtcgacaaggacggagacacaattgaatggggagaattccagatcgacggtagaagtgctagaggaggtcaacagacagctaacgactcatatgccaaggcgttaaacgcaacttctcttgaccaagcacttcaaatattgaaggaagaacaaccaaaggattacttccttcaacatcacaatcttttgaacaatgctcaaaagatatttcagaggccacctgatccatggactccactatttcctctgtcctcattcacaaacgttcctgaggaaatgcaagaatgggct

SEQ ID NO: 4 - DNA, 440 bp, Beet severe curly top virus
aaaatggggaaccacatctgcatgcccttattcaattcgaaggaaaagtccagatccgtaatgcccgttacttcgatctgcaacatcgaagtaccagcaaacaattccactgcaatattcagggagctaaatccagttccgacgtcaagtcctacgtctcaaaggacggagatcacatcgactggggtgaatttcaggtcgatggaagatctgcacgcggaggtcaacagacggctaatgatgctgcagcagaggcattaaatgcaggtaatgcattagaagctctgcagataataagggagaaactcccagaaaaatatatttttcagtatcacaacctcaaacctaacttagaggctatttttcttcctcctccagatctttatcaaccaccttttcctctttcttctttcactagagttccagaaattattcaagag

SEQ ID NO: 5 - DNA, 568 bp, Beet necrotic yellow vein virus
tcgagtgaaggtagatatatgacatggaaggatatgtcacataataagtttatgaccgatcgatgggcccgtgtttcggacgtcgtgagtgttattaaacaatcgcatgctatggatttgtccaaggctgcgaatctatctataattaaaactgctttggcaggattaggctcgggttggactgacaataatccttttgtgtccccgatgacccgttttccacagacactaactatgtacggtgcacttgtgttatatgttaatctgtccgacccagaatttgcgttgataatgactaaggtaagtactttaattgattcagggttagcagataatgcatctgctaatgtgcgtagagatgtggtgtccggaaataaagccgaatcatccggtaaaactgctggcactaatgagaattctgcttatacgcttaccgttagtcttgctggtttggctcaagctcttaggcttgaggaattaatgtggactcgggataagtttgaggaccggttgaagttaccatggacacctgttcaaggtagaaccagtccacccggacaatagcaat

(19) United States
(12) Patent Application Publication
(10) Pub. No.: US 2023/0183733 A1
(43) Pub. Date: Jun. 15, 2023

(54) MULTIPLE VIRUS RESISTANCE

(71) Applicant: KWS SAAT SE & Co. KGaA, Einbeck (DE)

(72) Inventors: Katia SCHUTZE, Einbeck (DE); Nadine SCHUMANN, Einbeck (DE); Josef KRAUS, Einbeck (DE)

(73) Assignee: KWS SAAT SE & Co. KGaA, Einbeck (DE)

(21) Appl. No.: 17/926,375

(22) PCT Filed: May 20, 2021
(86) PCT No.: PCT/EP2021/063490

§ 371 Date: Nov. 18, 2022

(30) Foreign Application Priority Data
May 20, 2020 (EP) 20175671.5

(51) Int. Cl. C12N 15/82

(52) U.S. Cl. CPC C12N 15/8283; C12N 15/8218

(57) ABSTRACT

The present invention relates to nucleic acid constructs or sets of nucleic acid constructs, which confer resistance to two or more viruses to plants of the genus Beta. The nucleic acid constructs comprise or encode sense and/or antisense sequences targeting a genomic sequence of at least two relevant viruses, which infect Beta plants. The present invention also provides transgenic Beta plants, which are resistant to at least two viruses as well as a method of conferring resistance to a Beta plant to at least two viruses, which comprises the step of transforming a Beta plant cell with a nucleic acid construct or a set of nucleic acid constructs according to the invention.

### TECHNICAL FIELD

The present invention relates to novel means and methods to provide plants, in particular of genus Beta, which are resistant to multiple viruses. The present invention provides nucleic acid constructs or sets of nucleic acid constructs, which confer resistance to two or more viruses to plants of the genus Beta. The nucleic acid constructs comprise or encode sense and/or antisense sequences targeting a genomic sequence of at least two relevant viruses, which infect Beta plants. The present invention also provides transgenic Beta plants, which are resistant to at least two viruses as well as a method of conferring resistance to a Beta plant to at least two viruses, which comprises the step of transforming a Beta plant cell with a nucleic acid construct or a set of nucleic acid constructs according to the invention.

### BACKGROUND

Plant viruses represent an ongoing problem in the production of many crops. Plants of the genus Beta, such as sugar beet plants, are susceptible to a number of viruses, which cause significant losses in yield if infection occurs during the cultivation period. Some of the viruses are transmitted by insects, others are soil borne and are transmitted by the protist Polymyxa betae. Against the aphid borne viruses, protection is often provided by using insecticides, e.g. as a seed coating. However, it is not desirable to apply chemicals on fields and/or plants as this may lead to a contamination of soil and water. Further, due to the ban of an increasing number of insecticides, the chemical control of several insects that act as a vector for plant viruses, becomes increasingly difficult and sometimes even impossible. In case of infection with aggressive pathotypes of Beet Necrotic Yellow Vein Virus (BNYVV), the loss of yield can be up to 90 percent and for this soil borne pathogen there is no direct chemical protection available. Infection with Beet Curly Top Virus (BCTV), Beet Severe Curly Top Virus (BSCTV) or yellowing viruses can reduce harvest in sugar beet by up to 50%. Conventional genetic resistances that sufficiently protect sugar beet against virus infection are only known for BNYVV (Rz1 and Rz2). For other sugar beet viruses, only partial resistance or tolerance is described. For BNYVV and Beet Mild Yellowing Virus (BMYV) transgenic resistances, which prevent infection and viral replication are described (WO2007/128755A1, WO2010/076212A1, EP2723873B2). Table 1 provides an overview of the economically most relevant Beta infecting viruses.

TABLE 1
Overview of Beta infecting viruses

| | Family | Genus |
|---|---|---|
| Beet Curly Top Virus (BCTV) | Geminiviridae | Curtovirus |
| Beet Severe Curly Top Virus | | |
| (BSCTV) | | |
| Beet Necrotic Yellow Vein Virus | Unassigned | Benyvirus |
| (BNYVV) | | |
| Beet Yellows Virus (BYV) | Closteroviridae | Closterovirus |
| Beet Mild Yellowing Virus (BMYV) | Luteoviridae | Polerovirus |
| Beet Western Yellowing Virus | | |
| (BWYV) | | |
| Beet Chlorosis virus (BChV) | | |

Systematic https://viralzone.expasy.org/

Transgenic means of protecting plants against multiple plant viruses are described, for example for tomatoes. Prins at al. (Journal of General Virology, 2006, 87, 3697-3701) in this regard describe a single transgene construct, which provides multiple virus resistance in tomato plants, i.e. against the four major tomato-infecting tospoviruses, Tomato Spotted Wilt Virus (TSWV), Groundnut Ringspot Virus (GRSV), Tomato Chlorotic Spot Virus (TCSV) and Watermelon Silver Mottle Virus (WSMoV).

U.S. Pat. No. 8,455,716B also relates to tomato plants, which are resistant to multiple viral strains. Resistance is provided by virus-derived sequences with at least two different modes of action selected from the group consisting of a dsRNA or miRNA being complementary to all or part of a target gene of said plant virus species and inhibition of tospovirus virion assembly.

However, for Beta plants, there are currently no means available (neither transgenic resistance nor natural resistances) to confer resistance to a combination of the viruses described above and conventional genetic diversity does not provide sufficiently high levels of resistance. In particular, no combined transgenic virus resistance to two or more of the mentioned viruses has been described. Ideally the crop plants should be broadly resistant to all relevant viruses, but even a resistance to a subset of the viruses represents an advantage in view of the prior art.

It was an object of the present invention to provide techniques for conferring resistance against at least two, preferably more than two viruses to a Beta plant. Particularly desirable is a resistance against all the relevant Beta infecting viruses mentioned above. The resistance to each virus should be stable and ideally the combined resistances should be heritable to following generations of Beta plants. Therefore, the introduction of resistances against different viruses in one genetic locus in the Beta genome would have a great benefit for the further use in breeding since inheritance of these traits would be coupled and could thus easily be transmitted to the next generation.

### SUMMARY OF THE INVENTION

In a first aspect, the present invention provides a nucleic acid construct or a set of nucleic acid constructs conferring resistance to a Beta plant to two or more of the viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV), wherein the nucleic acid construct or the set of nucleic acid constructs comprises or encodes at least one sense and/or antisense sequence targeting a genomic sequence of each of at least two viruses, or more than two viruses, selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV) Beet Western Yellowing Virus (BWYV) and Beet Chlorosis Virus (BChV) or orthologue sequences thereof.

In one embodiment of the various aspects of the present invention, the nucleic acid construct or the set of nucleic acid constructs described above comprises or encodes at least one sense and/or antisense sequence targeting a genomic sequence of each of at least three, at least four, at least five, at least six or all of the viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV), Beet Chlorosis Virus (BChV) or orthologue sequences thereof.

In another embodiment of the various aspects of the present invention, the nucleic acid construct or the set of nucleic acid constructs according to any of the embodiments described above comprises or encodes sense and/or antisense sequences targeting a genomic sequence of Beet Curly Top Virus (BCTV) and Beet Severe Curly Top Virus (BSCTV) or orthologue sequences thereof, or the nucleic acid construct or the set of nucleic acid constructs comprises or encodes sense and/or antisense sequences targeting a genomic sequence of Beet Curly Top Virus (BCTV), Beet Severe Curly Top Virus (BSCTV) and Beet Necrotic Yellow Vein Virus (BNYVV) or orthologue sequences thereof, or the nucleic acid construct or the set of nucleic acid constructs comprises or encodes sense and/or antisense sequences targeting a genomic sequence of Beet Curly Top Virus (BCTV), Beet Severe Curly Top Virus (BSCTV), Beet Necrotic Yellow Vein Virus (BNYVV), Beet Yellows Virus (BYV) and Beet Mild Yellowing Virus (BMYV) or orthologue sequences thereof or the nucleic acid construct or the set of nucleic acid constructs comprises or encodes sense and/or antisense sequences targeting a genomic sequence of Beet Curly Top Virus (BCTV), Beet Severe Curly Top Virus (BSCTV), Beet Necrotic Yellow Vein Virus (BNYVV), Beet Yellows Virus (BYV) and Beet Western Yellowing Virus (BWYV) or orthologue sequences thereof.

In a further embodiment of the various aspects of the present invention, in the nucleic acid construct or set of nucleic acid constructs according to any of the embodiments described above, the sense and/or antisense sequence(s) each have a length of at least 20 nt, at least 25 nt, at least 30 nt, at least 40 nt, at least 50 nt, at least 60 nt, at least 70 nt, at least 80 nt, at least 90 nt, at least 100 nt, at least 110 nt, at least 120 nt, at least 130 nt, at least 140 nt, at least 150 nt, at least 160 nt, at least 170 nt, at least 180 nt, at least 190 nt, at least 200 nt, at least 220 nt, at least 250 nt, at least 270 nt or at least 300 nt and/or the sense and/or antisense sequence(s) each have a length of 4000 nt or less, 3500 nt or less, 3000 nt or less, 2000 nt or less or 1000 nt or less.

In one embodiment of the various aspects of the present invention, in the nucleic acid construct or set of nucleic acid constructs according to any of the embodiments described above, the sense and/or antisense sequence(s) each have a length of 50 nt to 2000 nt, preferably 100 nt to 1000 nt, more preferably 300 nt to 600 nt.

In another embodiment of the various aspects of the present invention, in the nucleic acid construct or set of nucleic acid constructs according to any of the embodiments described above, the nucleic acid construct or the set of nucleic acid constructs comprises or encodes at least two corresponding sense and antisense sequences, wherein the sense sequences are selected from the sequences of SEQ ID NO: 1, SEQ ID NO: 2, SEQ ID NO: 3, SEQ ID NO: 4 and SEQ ID NO: 5 or fragments thereof or sequences having a sequence identity of at least 80%, at least 85%, at least 90%, at least 95% to the sequences of SEQ ID NO: 1, SEQ ID NO: 2, SEQ ID NO: 3, SEQ ID NO: 4 and SEQ ID NO: 5 or fragments thereof.

In yet another embodiment of the various aspects of the present invention, in the nucleic acid construct or set of nucleic acid constructs according to any of the embodiments described above, the nucleic acid construct or the set of nucleic acid constructs comprises or encodes at least the sequences of SEQ ID NO: 3 and SEQ ID NO: 4 or fragments thereof or sequences having a sequence identity of at least 80%, at least 85%, at least 90%, at least 95% to the sequences of SEQ ID NO: 3 and SEQ ID NO: 4 or fragments thereof and the corresponding antisense sequences, or the nucleic acid construct or the set of nucleic acid constructs comprises or encodes at least the sequences of SEQ ID NO: 3, SEQ ID NO: 4 and SEQ ID NO: 5 or fragments thereof or sequences having a sequence identity of at least 80%, at least 85%, at least 90%, at least 95% to the sequences of SEQ ID NO: 3, SEQ ID NO: 4 and SEQ ID NO: 5 or fragments thereof and the corresponding antisense sequences, or the nucleic acid construct or the set of nucleic acid constructs comprises or encodes at least the sequences of SEQ ID NO: 1, SEQ ID NO: 2, SEQ ID NO: 3, SEQ ID NO: 4 and SEQ ID NO: 5 or fragments thereof or sequences having a sequence identity of at least 80%, at least 85%, at least 90%, at least 95% to the sequences of SEQ ID NO: 1, SEQ ID NO: 2, SEQ ID NO: 3, SEQ ID NO: 4 and SEQ ID NO: 5 or fragments thereof and the corresponding antisense sequences.

In a further embodiment of the various aspects of the present invention, in the nucleic acid construct or set of nucleic acid constructs according to any of the embodiments described above, the nucleic acid construct or the set of nucleic acid constructs comprises or encodes the corresponding sense and antisense sequences arranged as follows:
(a) the sense sequences targeting all of the seven viruses recited above, six of the viruses recited above, five of the viruses recited above, four of the viruses recited above, three of the viruses recited above or two of the viruses recited above are arranged in contiguous order and the corresponding antisense sequences are arranged in the reverse contiguous order, or
(b) the sense sequences targeting five and two of the viruses recited above or four and three of the viruses recited above are arranged in contiguous order, respectively, and the corresponding antisense sequences are arranged in the reverse contiguous order, or
(c) the sense sequences targeting five of the viruses recited above are arranged in contiguous order and the corresponding antisense sequences are arranged in the reverse contiguous order while the sense sequences targeting the remaining two viruses recited above and the corresponding antisense sequences are not arranged in contiguous order, or the sense sequences targeting four and two of the viruses recited above are arranged in contiguous order, respectively, and the corresponding antisense sequences are arranged in the reverse contiguous order, or the sense sequences targeting three and three of the viruses recited above are arranged in contiguous order, respectively, and the corresponding antisense sequences are arranged in the reverse contiguous order, or the sense sequences targeting three, two and two of the viruses recited above are arranged in contiguous order, respectively, and the corresponding antisense sequences are arranged in the reverse contiguous order, or
(d) the sense sequences targeting two, two and two of the viruses recited above are arranged in contiguous order, respectively, and the corresponding antisense sequences are arranged in the reverse contiguous order, or the sense sequences targeting three and two of the viruses recited above are arranged in contiguous order, respectively, and the corresponding antisense sequences are arranged in the reverse contiguous order while the sense sequences targeting the remaining two viruses recited above and the corresponding antisense sequences are not arranged in contiguous order, or the sense sequences targeting four of the viruses recited above are arranged in contiguous order and the corresponding antisense sequences are arranged in the reverse contiguous order while the sense sequences targeting the remaining three viruses recited above and the corresponding antisense sequences are not arranged in contiguous order, or
(e) the sense sequences targeting two and two of the viruses recited above are arranged in contiguous order, respectively, and the corresponding antisense sequences are arranged in the reverse contiguous order while the sense sequences targeting the remaining three viruses recited above and the corresponding antisense sequences are not arranged in contiguous order, or the sense sequences targeting three of the viruses recited above are arranged in contiguous order and the corresponding antisense sequences are arranged in the reverse contiguous order while the sense sequences targeting the remaining four viruses recited above and the corresponding antisense sequences are not arranged in contiguous order, or
(f) the sense sequences targeting two of the viruses recited above are arranged in contiguous order and the corresponding antisense sequences are arranged in the reverse contiguous order while the sense sequences targeting the remaining five viruses recited above and the corresponding antisense sequences are not arranged in contiguous order, or
(g) none of the sense sequences targeting the seven viruses recited above or the corresponding antisense sequences are arranged in contiguous order.

In one embodiment, in the nucleic acid construct or set of nucleic acid constructs according to the embodiment described above, the sense sequences are selected from the sequences of SEQ ID NO: 1, SEQ ID NO: 2, SEQ ID NO: 3, SEQ ID NO: 4 and SEQ ID NO: 5 or fragments thereof or sequences having a sequence identity of at least 80%, at least 85%, at least 90%, at least 95% to the sequences of SEQ ID NO: 1, SEQ ID NO: 2, SEQ ID NO: 3, SEQ ID NO: 4 and SEQ ID NO: 5 or fragments thereof.

In another aspect, the present invention relates to an RNA molecule or a set of RNA molecules, which is/are formed upon transcription of a nucleic acid construct or set of nucleic acid constructs according to any of the embodiments described above.

In a further aspect, the present invention relates to a vector or set of vectors comprising a nucleic acid construct or set of nucleic acid constructs according to any of the embodiments described above.

In yet another aspect, the present invention relates to a virus resistant transgenic Beta plant, cell of a Beta plant, part of a Beta plant or seed of a Beta plant comprising a nucleic acid construct or set of nucleic acid constructs according to any of the embodiments described above, or comprising an RNA molecule or set of RNA molecules according to the embodiment described above or comprising a vector or set of vectors according to the embodiment described above.

In one aspect, the present invention relates to a method of conferring resistance to a Beta plant to at least two viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV) comprising the steps:
(i) transforming at least one Beta plant cell with a nucleic acid construct or set of nucleic acid constructs according to any of the embodiments described above or a vector or set of vectors according to the embodiment described above, and
(ii) regenerating a Beta plant from the transformed cell of step (i),
(iii) optionally, selecting and/or obtaining a Beta plant, which is resistant to at least two viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV) Beet Western Yellowing Virus (BWYV) and Beet Chlorosis Virus (BChV).

In one embodiment of the method described above,

in step (i) a first Beta plant cell is transformed with a nucleic acid construct or a set of nucleic acid constructs comprising sense and/or antisense sequences targeting a genomic sequence of each of two, three, four or five viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV) or orthologous sequences thereof and a second Beta plant cell is transformed with a nucleic acid construct or a set of nucleic acid constructs comprising sense and/or antisense sequences targeting a genomic sequence of each of two, three, four or five of the viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV) or orthologous sequences thereof, and

in step (ii) a first Beta plant is regenerated from the transformed first Beta plant cell and a second Beta plant is regenerated from the transformed second Beta plant cell, and

in step (iii) a first Beta plant is selected and/or obtained, which is resistant to two, three, four or five of the viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV) Beet Western Yellowing Virus (BWYV) and Beet Chlorosis Virus (BChV) and a second Beta plant is selected and/or obtained, which is resistant to two, three, four or five of the viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV), and

the method comprises a step (iv) of crossing the first and the second plant selected and/or obtained in step (iii) to generate an F1 generation of the plants and, optionally,

a step (v) of obtaining and/or selecting a plant, which is resistant to at least four, five, six or all of the viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV).

In another aspect, the present invention relates to a use of a nucleic acid construct or a set of nucleic acid constructs according to any of the embodiments described above to confer resistance to at least two viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV) to a Beta plant.

### Definitions

A “nucleic acid construct” refers to a nucleic acid molecule encoding or comprising one or more genetic elements, which upon introduction into a target cell can be transcribed and/or translated into a functional form, e.g. RNA(s) or polypeptide(s) or protein(s). A nucleic acid construct may also comprise regulatory sequences such as promoter and terminator sequences facilitating expression of the genetic element(s) as well as spacers and introns. The genetic elements of the present invention can also be encoded on a set of constructs, which constructs can be introduced into a cell simultaneously or consecutively.

A “regulatory element” or “regulatory sequence” refers to a nucleotide sequence which is not part of the protein-encoding nucleotide sequence but mediate the expression of the protein-encoding nucleotide sequence. Regulatory elements include, for example, promoters, cis-regulatory elements, enhancers, introns or terminators. Depending on the type of regulatory element, it is located on the nucleic acid molecule before (i.e., 5′ of) or after (i.e., 3′ of) the protein-encoding nucleotide sequence. The regulatory elements are functional in a living plant cell.

The term “operatively linked” means that a regulatory element is linked in such a way with the protein-encoding nucleotide sequence, i.e. is positioned in such a way relative to the protein-encoding nucleotide sequence on, for example, a nucleic acid molecule that an expression of the protein-encoding nucleotide sequence under the control of the regulatory element can take place in a living cell.

A “promoter” refers to a DNA sequence capable of controlling and/or regulating expression of a coding sequence, i.e. a gene or part thereof, or of a functional RNA, i.e. a RNA which is active without being translated, for example, a miRNA, a siRNA, an inverted repeat RNA or a hairpin forming RNA. A promoter is usually located at the 5′ part of a sequence to be transcribed and mediates the start of transcription by RNA polymerase by interaction with certain DNA-binding proteins. Examples of promoters which are functional in plant cells, include constitutive promoters such as viral promoters, for example, the CaM35S promoter, a double CaM35S promoter, or plant promoters such as the ubiquitin promoters as described in EP 0 305 668 and U.S. Pat. No. 6,528,701. Furthermore, promoters may be used, which have, for example, specific activity at certain stages of development or which are inducible by environmental factors such as biotic or abiotic stress, or which are tissue-specific. Such promoters are known from the art.

Furthermore, suitable promoters include synthetic promoters. These are promotors that have been created by molecular biology techniques that are not found in nature in this configuration. A synthetic promoter is a minimalistic promoter containing only one or more selected, defined cis-elements in addition to a minimal promoter. These cis-elements are binding sites for DNA-binding proteins such as transcription factors and are isolated from natural promoters, derived from previously isolated cis-elements, or produced technically by random recombination techniques and selected by appropriate methods; as compared with a natural promoter, due to its less complex construction a synthetic promoter is activated only by a few exogenous and endogenous factors and is therefore more specifically regulated.

The “minimal promoter” or “core”-promoter is a nucleotide sequence which contains the binding sites for the basal transcription factor complex and allows the accurate initiation of transcription by RNA polymerase II. Characteristic sequence motifs of the minimal promoter are the TATA box, the initiator element (lnr), the “TFBII recognition element” (BRE) and the “downstream core promoter element” (OPE). In the minimal promoter these elements can occur individually or in combination. The minimal promoter or its sequence motifs are available, for example, from any plant, bacterial, fungal or viral gene.

“Cis elements” are nucleotide sequences that are located on the same nucleic acid molecule as the protein-encoding nucleotide sequence to be expressed. Cis elements do not have to encode RNA or protein and in the direction of transcription can be located before or after the protein-encoding nucleotide sequence to be expressed. Cis elements upstream before a protein-encoding nucleotide sequence to be expressed often provide necessary binding motifs in particular for transcription factors which engage as trans-acting elements (of Lat. trans, ‘beyond’), on the molecular level, from the other side in the regulation of the transcription of this gene. If, in addition, cis elements lead to an inhibition of the transcription, they are called silencers. Cis elements that lead to an enhancement of the transcription are called enhancers. The totality of the cis/trans activities in the promoter determines the intensity with which the RNA polymerase carries out transcription.

Furthermore, a promoter may be a chimeric promoter and/or a promoter that has been modified by cis elements. The modification of a promoter can also mean the additional incorporation of a cis element in the promoter which for example already has a cis-element naturally. Further, the modification also includes a multimerization of a cis element, in particular a multimerization of a naturally existing cis element. Compared with the native version such modified promoter may have altered properties with respect to specificity, expression level or background activity, for example.

A “terminator” is a DNA sequence that mediates transcriptional termination, i.e. the release of the transcript RNA from the transcriptional complex. The terminator is usually located at the 3′ end of a sequence to be transcribed.

A plant exhibits “resistance” to a pant virus, when symptoms of virus infection are reduced or not observed at all when the plant is exposed to the virus under conditions allowing infection. In particular, a resistant plant shows reduced yield loss compared to a non-resistant plant infected with the same virus under the same conditions. Ideally, in a resistant plant, the yield loss caused by virus infestation is completely compensated, meaning that the plant produces as much yield as a plant, which was not exposed to the virus at all but grown under the same conditions. How much yield loss is caused by a virus in a non-resistant plant depends on several factors including the virus itself and the stage of plant development, in which the infection occurs. Yellowing viruses such as BMYV and BYV typically cause up to 50% yield loss, BCTV and BSCTV up to 30% and BNYVV up to 90%. Further, resistant plants show decreased viral replication within the plants and thus a decrease in virus content. In a resistant plant, the virus titer is typically reduced by up to 50%, up to 70% or up to 90% with respect to a non-resistant plant infected with the virus under the same conditions.

“Sense” and an “antisense” sequences are complementary sequences, which are present in reverse orientation in a nucleic acid sequence. If a nucleic acid construct of the present invention comprises a sense and a corresponding antisense sequence, the two complementary sequences form an RNA double strand upon transcription, which may be (part of) an RNA hairpin structure. In an RNA hairpin structure, there can be several sense sequences and corresponding antisense sequences, which together form a double strand and are separated by an intron forming the loop of the hairpin structure. Two or more sense sequences may be arranged in “contiguous order”, i.e. in a sequential arrangement, which is followed by an intron sequence, and then the corresponding antisense sequences in the reverse contiguous order. In such an arrangement, the sense and antisense sequences form one double strand upon transcription, with the intron forming a loop. Furthermore, two or more hairpin structures, each comprising one or more contiguous sense sequences and the corresponding antisense sequences in reverse contiguous order may be present on one nucleic acid construct separated by spacer sequences.

A “fragment” of a sequence in the context of the present invention refers to any contiguous part of the sequence as identified by the SEQ ID NO. A fragment is preferably at least 20 nt, at least 25 nt, at least 30 nt, at least 40 nt, at least 50 nt, at least 60 nt, at least 70 nt, at least 80 nt, at least 90 nt, at least 100 nt, at least 110 nt, at least 120 nt, at least 130 nt, at least 140 nt, at least 150 nt, at least 160 nt, at least 170 nt, at least 180 nt, at least 190 nt, at least 200 nt, at least 220 nt, at least 250 nt, at least 270 nt or at least 300 nt long.

A sequence comprised or encoded by a nucleic acid construct of the present invention “targets” a genomic sequence when it contains sequence information, which allows recognition of the genomic sequence and can thus interfere with the sequence, e.g. by site-specific cleavage or silencing. The targeting can be affected either by direct interaction with the genomic sequence itself or by interaction with the transcript of the genomic sequence. For example, if the nucleic acid construct of the present invention comprises or encodes a sense and a corresponding antisense sequence targeting a genomic sequence, an RNA silencing or RNA interference (RNAi) mechanism is activated upon transcription of the construct, which leads to the destruction of the transcript of the genomic target sequence and thus suppresses expression of the target. In another case, the nucleic acid construct may encode a nucleic acid guided nuclease and a guide RNA, which results in cleavage of the target sequence.

The terms “RNA interference” or “RNAi” or “RNA silencing” as used herein interchangeably refer to a gene down-regulation (or knockdown) mechanism meanwhile demonstrated to exist in all eukaryotes. The mechanism was first recognized in plants where it was called “post-transcriptional gene silencing” or “PTGS”. In RNAi, small RNAs function to guide specific effector proteins to a target nucleotide sequence by complementary base pairing resulting in degradation of the target.

A “nucleic acid guided nuclease” is a site-specific nuclease, which requires a nucleic acid molecule, in particular a guide RNA, to recognize and cleave a specific target site, e.g. in genomic DNA. The nucleic acid guided nuclease forms a nuclease complex together with the guide nucleic acid and then recognizes and cleaves the target site in a sequence-dependent matter. Nucleic acid guided nucleases can therefore be programmed to target a specific site by the design of the guide nucleic acid sequence. Examples are the nucleases of CRISPR/Cas systems, CRISPR/Cas12a systems, CRISPR/Cas13 systems, CRISPR/CasX systems, CRISPR/CasY systems, CRISPR/Cmr systems, CRISPR/MAD7_systems, CRISPR/MAD2 systems and/or any combination, variant, or catalytically active fragment thereof. The “guide RNA” may be a trans-activating CRISPR RNA (tracrRNA) plus a synthetic CRISPR RNA (crRNA) or a single guide RNA (sgRNA), which comprises the sequence information targeting the genomic sequence for cleavage by the nuclease.

“Orthologous sequences” are genomic sequences in different species, which share a certain sequence similarity and can be traced back to a common ancestral sequence. They are the result of a speciation event, where a species diverged into two separate species. Orthologous sequences often have the same function but may also have different functions.

The term “vector” refers to an element used for introducing the nucleic acid construct or the set of nucleic acid constructs according to the invention into a cellular system. The vector may be a plasmid or plasmid vector, cosmid, artificial yeast artificial chromosomes (YAC), bacterial artificial chromosome (BAC) or Pl artificial chromosomes (PACs), phagemid, bacterial phage based vector, an isolated single-stranded or double-stranded nucleic acid sequence, comprising DNA and RNA sequences in linear or circular form, or a mixture thereof, for introduction or transformation into a plant, plant cell, tissue, organ or material according to the present disclosure.

“Transforming” a plant cell with the construct or set of constructs according to the invention refers to any established technique to introduce nucleic acid molecules into a cell, such as biolistic approaches (e.g. particle bombardment), microinjection, permeabilising the cell membrane with various treatments such as electroporation or PEG treatment or Agrobacterium tumefaciens mediated transformation. Generally, incorporating the nucleic acid construct(s), for example by way of transformation, may be accomplished with techniques that are basically known to the person skilled in the art. For example, the nucleic acid construct can be incorporated into the plant cells by infecting a plant tissue or a plant cell with Agrobacterium tumefaciens containing the nucleic acid sequence to be transferred in its plasmid that can be integrated into the plant genome. Incorporating by means of a biolistic transfer is another option, wherein the nucleic acid construct to be incorporated into the plant cell is applied to gold particles or tungsten particles, which are then shot into the cells at a high speed. Another option known to the person skilled in the art for incorporating the nucleic acid construct into a plant cell, is the protoplast transformation, wherein either polyethylene glycol is added to the protoplasts in the presence of the nucleic acid molecules to be incorporated, or the protoplasts are exposed to a short current impulse, so that the protoplast membrane transiently becomes permeable for the nucleic acid construct(s). Methods for regenerating whole plants from transformed tissue or cells are also known to the person skilled in the art from the prior art.

Preferably, the nucleic acid construct or set of nucleic acid constructs according to the invention are stably incorporated into the genome of the cell of the plant. This means following regeneration of a plant the transferred nucleic acid sequence may be stably passed from this plant to a progeny plant.

Preferably, the transformation and regeneration of sugar beet is carried out by the method described by Lindsey (Lindsey K. (1991) “Regeneration and transformation of sugar beet by Agrobacterium tumefaciens” Plant Tissue Culture Manual B7: 1-13, Kluwer Academic Publishers). Further preferred is Agrobacterium mediated transformation as described by in Kishchenko et al., Production of transgenetic sugarbeet (Beta vulgaris L.) plants resistant to phosphinothricin (2005) Cell Biology International, 29(1): 15-19.

The transgenesis of the plants can be verified by polymerase chain reaction using appropriate oligonucleotide primers. After regeneration, the transformants can be grown and selfed for obtaining seeds in the greenhouse.

“Targeted integration” means targeted integration of exogenous sequences into a region of interest in the genome of a Beta cell. Targeted integration of an exogenous sequence occurs at a double-strand break in the Beta genome by both homology-dependent and homology-independent mechanisms. Targeted integration by both homology-dependent and homology-independent mechanisms may involve insertion of an exogenous sequence between the ends generated by cleavage. The exogenous sequence inserted can be of any length. In embodiments in which targeted integration occurs by a homology-dependent mechanism, the donor sequence contains sufficient homology, in the regions flanking the exogenous sequence, to support homology-directed repair of a double-strand break in a genomic sequence, thereby inserting the exogenous sequence at the genomic target site. Therefore, the donor nucleic acid can be of any size sufficient to support integration of the exogenous sequence by homology-dependent repair mechanisms (e.g., homologous recombination).

“Landing sites” as used herein, refers to a DNA sequence within the genome of a Beta species capable of being targeted by an endonuclease complex. Such a landing site generally comprises a specific combination of motif sequence that is capable of being cleaved a given endonuclease complex. It is well known to the person of skill in the art to determine landing sites for a given endonuclease complex. CRISPR landing sites, for example, i.e. a DNA sequence capable of being targeted by a CRISPR complex, comprises a proximately placed protospacer/Protopacer Adjacent Motif (PAM) combination sequence that is capable of being cleaved a CRISPR endonuclease complex.

In the context of the present disclosure, a Beta plant is a plant of genus Beta. In particular, a Beta plant is a Beta vulgaris spp. plant, more specifically a Beta vulgaris subsp. vulgaris plant. The terms “plant” or “plant cell” or “part of a plant” as used herein refer to a plant organism, a plant organ, differentiated and undifferentiated plant tissues, plant cells, seeds, and derivatives and progeny thereof. Plant cells include without limitation, for example, cells from seeds, from mature and immature cells or organs, including embryos, meristematic tissues, seedlings, callus tissues in different differentiation states, leaves, flowers, roots, shoots, male or female gametophytes, sporophytes, pollen, pollen tubes and microspores and protoplasts.

A step of “crossing” a first and a second plant refers to a process where the first and the second plant are interbred to produce a progeny, i.e. an “F1 generation”, which comprises genes of both, the first and the second plant, mixed by homologous recombination. The F1 generation can be screened for a desired combination of the parental genes.

Nucleic acid sequences or nucleic acid molecules disclosed herein can be “codon-optimized”. “Codon optimization” implies that a DNA or RNA synthetically produced or isolated from a donor organism is adapted to the codon usage of different recipient organism to improve transcription rates, mRNA processing and/or stability, and/or translation rates, and/or subsequent protein folding of said recombinant nucleic acid in the cell or organism of interest. The skilled person is well aware of the fact that a target nucleic acid can be modified at one position due to the codon degeneracy, whereas this modification will still lead to the same amino acid sequence at that position after translation, which is achieved by codon optimization to take into consideration the species-specific codon usage of a target cell or organism. In turn, nucleic acid sequences as defined herein may have a certain degree of identity to a different sequence, encoding the same protein, but having been codon optimized.

Whenever the present disclosure relates to the percentage of identity of nucleic acid or amino acid sequences to each other these values define those values as obtained by using the EMBOSS Water Pairwise Sequence Alignments (nucleotide) programme (www.ebi.ac.uk/Tools/psa/emboss_water/nucleotide.html) nucleic acids or the EMBOSS Water Pairwise Sequence Alignments (protein) programme (www.ebi.ac.uk/Tools/psa/emboss_water/) for amino acid sequences. Alignments or sequence comparisons as used herein refer to an alignment over the whole length of two sequences compared to each other. Those tools provided by the European Molecular Biology Laboratory (EMBL) European Bioinformatics Institute (EBI) for local sequence alignments use a modified Smith-Waterman algorithm (see www.ebi.ac.uk/Tools/psa/and Smith, T. F. & Waterman, M. S. “Identification of common molecular subsequences” Journal of Molecular Biology, 1981 147 (1):195-197). When conducting an alignment, the default parameters defined by the EMBL-EBI are used. Those parameters are (i) for amino acid sequences: Matrix=BLOSUM62, gap open penalty=10 and gap extend penalty=0.5 or (ii) for nucleic acid sequences: Matrix=DNAfull, gap open penalty=10 and gap extend penalty=0.5.

In the context of the present invention, sequence identity is to be determined with respect to the full length of the respective sequence given under a SEQ ID NO or a fragment thereof, which has the same length as the sequence to be compared.

### BRIEF DESCRIPTION OF THE FIGURES

FIG. 1 shows a schematic representation of the viral genomes and the tested RNAi target regions. (A) Linearized BCTV/BSCTV genome and the targets V2/MP/CP; C2/C3 and C4/Rep; (B) Polerovirus (BMYV/BWYV/BChV) genome and the targets P0/P1; P3/P4; RNAi3; RNAi4 and RNAi5 (C) BYV genome and the targets CDS5, CDS6 and CDS7; (D) RNA2 of the BNYVV genome and the target CP.

FIG. 2 shows the results of the BYV infection experiment: The mean relative virus titer (BYV) was determined by ELISA. Shown are controls infected and noninfected wildtype (transformation genotype) and in comparison, infected independent transgenic lines containing an CDS7 hairpin construct. The plants were infected with BYV using aphids as vector. Leaf samples for virus quantification were taken 6 wpi.

FIG. 3 shows the results of the BMYV infection experiment: The mean relative virus titer (BMYV) was determined by ELISA. Shown are controls infected and noninfected wildtype (transformation genotype) and in comparison, infected independent transgenic lines containing an P3/P4 hairpin construct. The plants were infected with an infectious BMYV full-length clone using Agrobacteria infiltration and leaf samples for virus quantification were taken 5 wpi.

FIG. 4 shows the results of the BCTV infection experiment: The mean relative virus titer (BCTV) was determined by qPCR. Shown are controls infected and noninfected wildtype (transformation genotype) and in comparison, different independent transgenic lines containing an C4/Rep hairpin construct. The plants were infected with an infectious BCTV full-length clone using Agrobacteria infiltration and leaf samples for virus quantification were taken 4 wpi.

FIG. 5 shows the schematic designs of combination constructs comprising one hairpin under the control of one promoter, two hairpins under the control of one promoter, two hairpins under control of two promoters and three hairpins under control of three promoters. Variation of the promoters, the orientation and the arrangement of genetic elements are used.

FIG. 6 shows a schematic representation of the design of the hairpin construct (antisense (as)—intron—sense (s)) MVR004 containing a Cauliflower mosaic virus d35S promoter, BMYV_P3/P4_as, BYV_CDS7_as, BCTV_C4/Rep_as, BSCTV_C4/Rep_as, BNYVV_CP_as, StLS1 intron, BNYVV_CP_s, BSCTV_C4/Rep_s, BCTV_C4/Rep_s, BYV_CDS7_s, BMYV_P3/P4_s and a nosT terminator.

FIG. 7 shows a schematic representation of the design of the hairpin construct (antisense (as)—intron—sense (s)) MVR005 containing an Arabidopsis AtUbi10 promoter, BMYV_P3/P4_as, BYV_CDS7_as, BCTV_C4/Rep_as, BSCTV_C4/Rep_as, BNYVV_CP_as, StLS1 intron, BNYVV_CP_s, BSCTV_C4/Rep_s, BCTV_C4/Rep_s, BYV_CDS7_s, BMYV_P3/P4_s and a nosT terminator.

FIG. 8 shows a schematic representation of the design of the hairpin construct (antisense (as)—intron—sense (s) MVR006 containing a Parsley PcUbi4 promoter, BMYV_P3/P4_as, BYV_CDS7_as, BCTV_C4/Rep_as, BSCTV_C4/Rep_as, BNYVV_CP_as, StLS1 intron, BNYVV_CP_s, BSCTV_C4/Rep_s, BCTV_C4/Rep_s, BYV_CDS7_s, BMYV_P3/P4_s and a nosT terminator.

FIG. 9 shows a schematic representation of the design of the hairpin construct (sense (s)—intron—antisense (as)) MVR007 containing a Cauliflower mosaic virus d35S promoter, BMYV_P3/P4_s, BYV_CDS7_s, BCTV_C4/Rep_s, BSCTV_C4/Rep_s, BNYVV_CP_s, StLS1 intron, BNYVV_CP_as, BSCTV_C4/Rep_as, BCTV_C4/Rep_as, BYV_CDS7_as, BMYV_P3/P4_as and a nosT terminator.

FIG. 10 shows a schematic representation of the design of the hairpin construct (antisense (as)—intron—sense (s)_antisense—intron—sense) MVR008 containing a Cauliflower mosaic virus d35S promoter, BMYV_P3/P4_as, BYV_CDS7_as, BNYVV_CP_as, StLS1 intron, BNYVV_CP_s, BYV_CDS7_s, BMYV_P3/P4_s, spacer, BCTV_C4/Rep_as, BSCTV_C4/Rep_as, Arabidopsis AtAAP6 intron, BSCTV_C4/Rep_s, BCTV_C4/Rep_s and a nosT terminator.

FIG. 11 shows a schematic representation of the design of the hairpin construct (antisense (as)—intron—sense (s)_antisense—intron—sense) MVR009 containing an Arabidopsis AtUbi10 promoter, BMYV_P3/P4_as, BYV_CDS7_as, BNYVV_CP_as, StLS1 intron, BNYVV_CP_s, BYV_CDS7_s, BMYV_P3/P4_s, spacer, BCTV_C4/Rep_as, BSCTV_C4/Rep_as, Arabidopsis AtAAP6 intron, BSCTV_C4/Rep_s, BCTV_C4/Rep_s and a nosT terminator.

FIG. 12 shows a schematic representation of the design of the hairpin construct (antisense (as)—intron—sense (s)_antisense—intron—sense) MVR010 containing an Arabidopsis AtUbi10 promoter, BYV_CDS7_as, BMYV_P3/P4_as, Arabidopsis AtAAP6 intron, BMYV_P3/P4_s, BYV_CDS7_s, nosT terminator, a Cauliflower mosaic virus d35S promoter, BCTV_C4/Rep_as, BSCTV_C4/Rep_as, BNYVV_CP_as, StLS1 intron, BNYVV_CP_s, BSCTV_C4/Rep_s, BCTV_C4/Rep_s and a 35S terminator.

FIG. 13 shows a schematic representation of the design of the hairpin construct (antisense (as)—intron—sense (s)_antisense—intron—sense) MVR011 containing a Cauliflower mosaic virus d35S promoter, BNYVV_CP_as, BMYV_P3/P4_as, BYV_CDS7_as, Arabidopsis AtAAP6 intron, BYV_CDS7_s, BMYV_P3/P4_s, BNYVV_CP_s, 35S terminator, an Arabidopsis AtUbi10 promoter, BCTV_C4/Rep_as, BSCTV_C4/Rep_as, StLS1 intron, BSCTV_C4/Rep_s, BCTV_C4/Rep_s and a nosT terminator.

FIG. 14 shows a schematic representation of the design of the hairpin construct (antisense (as)—intron—sense (s)_antisense—intron—sense) MVR012 containing a Cauliflower mosaic virus d35S promoter, BNYVV_CP_as, BMYV_P3/P4_as, BYV_CDS7_as, Arabidopsis AtAAP6 intron, BYV_CDS7_s, BMYV_P3/P4_s, BNYVV_CP_s, 35S terminator, an Arabidopsis AtUbi10 promoter, BCTV_C4/Rep_as, BSCTV_C4/Rep_as, StLS1 intron, BSCTV_C4/Rep_s, BCTV_C4/Rep_s and a nosT terminator.

FIG. 15 shows results of a representative qRT-PCR assay of independent MVR005 transgenic lines (construct as shown in FIG. 7). The relative expression of the hairpin construct was analyzed using a SYBR system and nosT oligonucleotides and is displayed in the graph.

FIG. 16 shows results of a siRNA quantification from independent MVR005 transgenic lines (construct as shown in FIG. 7). The siRNA amount was determined by miRNA sequencing and mapping to the hairpin construct. The percentage of total siRNA mapping to the construct is displayed in the graph.

### DETAILED DESCRIPTION

The present invention establishes a new concept to confer multiple virus resistance to a Beta plant and provides transgenic Beta plants, which are resistant to two, several, or even all, of the economically most relevant Beta infecting viruses: Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV) Beet Western Yellowing Virus (BWYV) and Beet Chlorosis Virus (BChV). These viruses represent the major viral plant pests of plants of the genus Beta with the highest economic relevance for sugar beets. The viruses belong to different viral genera and families, respectively, as shown earlier in Table 1.

In a first aspect, the present invention relates to a nucleic acid construct or a set of nucleic acid constructs conferring resistance to a Beta plant to two or more of the viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV), wherein the nucleic acid construct or the set of nucleic acid constructs comprises or encodes at least one sense and/or antisense sequence targeting a genomic sequence of each of at least two viruses, or more than two viruses, selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV) Beet Western Yellowing Virus (BWYV) and Beet Chlorosis Virus (BChV) or orthologue sequences thereof.

For the targeting of a genomic sequence, there are two modes of action, namely interaction with the genomic sequence itself or with a transcript of the genomic sequence. The gemini viruses BCTV and BSCTV do not have RNA transcripts. They nick their circular DNA genome and use rolling circle amplification for direct DNA synthesis. In this case, genomic DNA is directly targeted. For the ssRNA viruses, on the other hand, both is possible. siRNAs can hybridize to transcripts but also to single strand genomic sequences.

It is also possible that one sense and/or antisense sequences targets two or more viruses, namely when the genomic targets in the two or more viruses are orthologous and/or have a sufficient sequence similarity.

The construct or set of constructs according to the present invention combines target regions of at least to viruses to generate multiple resistant transgenic plants. Thus, resistance to a combination of virus pests from the genus Polerovirus, Benyvirus, Curtovirus and Geminivirus can be achieved in one transformation step. The integration of the construct or constructs into one genomic locus of the Beta plant also allows a stable heritability of the combined resistances.

The resistance is achieved by introducing into a Beta plant a nucleic acid construct, which targets a genomic sequence of each of at least two of the viruses recited above. The nucleic acid construct comprises either a) a combination of a promoter, a sense and an antisense sequence derived from each of at least two of the viruses, an intron and a terminator, or the nucleic acid construct comprises b) a combination of a promoter, a nucleic acid guided nuclease such as, for example, Cas12a or Cas13, a terminator and guide RNAs such as crRNAs, which may be sense or antisense sequences recognizing complementary viral genomic sequences of at least two viruses. In case a), an RNA hairpin structure is formed upon transcription of the construct, which is processed to short interfering RNAs (shRNAs), and results in the recognition and subsequent degradation of the viral genomic targets by an RNAi or RNA silencing mechanism. In case b), a nucleic acid guided nuclease and guide RNAs are expressed from the nucleic acid construct leading to cleavage of the viral genomic targets by the nuclease, which is directed to the target by the guide RNAs. In both cases, the expression of the viral targets is suppressed or prevented, abolishing infection and virus replication. The resulting Beta plant exhibits reduced yield losses and disease symptoms in the presence of the viruses compared to conventional varieties.

The increase in the resistance may be achieved by integration of the nucleic acid construct or set of constructs according to the present invention into the genome of at least one cell of a plant of the species Beta. In a preferred embodiment, the integration is a targeted insertion of the construct or constructs at a specific location in the genome. Subsequently, a plant can be regenerated from the plant cell. The integration may take place both by means of transformation and subsequent selection, or by means of homology-directed repair or homologous recombination. The two latter methods cited are preferably supported by site-directed nucleases which may be selected from, but are not limited to, the following: CRISPR nuclease, including Cas9, CasX, CasY, or Cpf1 nuclease, TALE nuclease, zinc finger nuclease, meganuclease, Argonaut nuclease, restriction endonuclease, including FokI or a variant thereof, recombinase, or two, site-specific, nicking endonucleases. Site-directed nucleases recognize certain landing sites in a DNA sequence within the genome, at which cleavage and insertion of an exogenous sequence takes place.

In one embodiment, the nucleic acid construct or set of nucleic acid constructs described above comprises or encodes at least one nucleic acid guided nuclease, preferably a Cas12a or Cas13, and at least one guide RNA targeting a genomic sequence of each of at least two viruses, or more than two viruses, selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV) Beet Western Yellowing Virus (BWYV) and Beet Chlorosis Virus (BChV) or orthologue sequences thereof.

The nucleic acid construct or set of constructs according to the present invention may be designed to target any subset of the recited viruses depending on specific, e.g. local, requirements. Thus, resistance against any relevant combinations of the above viruses can be obtained.

In one embodiment of the nucleic acid construct or a set of nucleic acid constructs described above, the nucleic acid construct or the set of nucleic acid constructs comprises or encodes at least one sense and/or antisense sequence targeting a genomic sequence of each of at least three, at least four, at least five, at least six or all of the viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV), Beet Chlorosis Virus (BChV) or orthologue sequences thereof.

In another embodiment of the nucleic acid construct or a set of nucleic acid constructs described above, the nucleic acid construct or the set of nucleic acid constructs comprises or encodes sense and/or antisense sequences targeting a genomic sequence of Beet Curly Top Virus (BCTV) and Beet Severe Curly Top Virus (BSCTV) or orthologue sequences thereof, or the nucleic acid construct or the set of nucleic acid constructs comprises or encodes sense and/or antisense sequences targeting a genomic sequence of Beet Curly Top Virus (BCTV), Beet Severe Curly Top Virus (BSCTV) and Beet Necrotic Yellow Vein Virus (BNYVV) or orthologue sequences thereof, or the nucleic acid construct or the set of nucleic acid constructs comprises or encodes sense and/or antisense sequences targeting a genomic sequence of Beet Curly Top Virus (BCTV), Beet Severe Curly Top Virus (BSCTV), Beet Necrotic Yellow Vein Virus (BNYVV), Beet Yellows Virus (BYV) and Beet Mild Yellowing Virus (BMYV) or orthologue sequences thereof or the nucleic acid construct or the set of nucleic acid constructs comprises or encodes sense and/or antisense sequences targeting a genomic sequence of Beet Curly Top Virus (BCTV), Beet Severe Curly Top Virus (BSCTV), Beet Necrotic Yellow Vein Virus (BNYVV), Beet Yellows Virus (BYV) and Beet Western Yellowing Virus (BWYV) or orthologue sequences thereof.

In order to achieve sufficient target specificity and at the same time to provide constructs of a manageable size, each one of the sense and/or antisense sequences should preferably have a length within a certain range.

In one embodiment of the nucleic acid construct or set of nucleic acid constructs described above, the sense and/or antisense sequence(s) each have a length of at least at least 20 nt, at least 25 nt, at least 30 nt, at least 40 nt, at least 50 nt, at least 60 nt, at least 70 nt, at least 80 nt, at least 90 nt, at least 100 nt, at least 110 nt, at least 120 nt, at least 130 nt, at least 140 nt, at least 150 nt, at least 160 nt, at least 170 nt, at least 180 nt, at least 190 nt, at least 200 nt, at least 220 nt, at least 250 nt, at least 270 nt or at least 300 nt and/or the sense and/or antisense sequence(s) each have a length of 4000 nt or less, 3500 nt or less, 3000 nt or less, 2000 nt or less or 1000 nt or less.

In another embodiment of the nucleic acid construct or set of nucleic acid constructs described above, the sense and/or antisense sequence(s) have a length of 50 nt to 2000 nt, preferably 100 nt to 1000 nt, more preferably 300 nt to 600 nt.

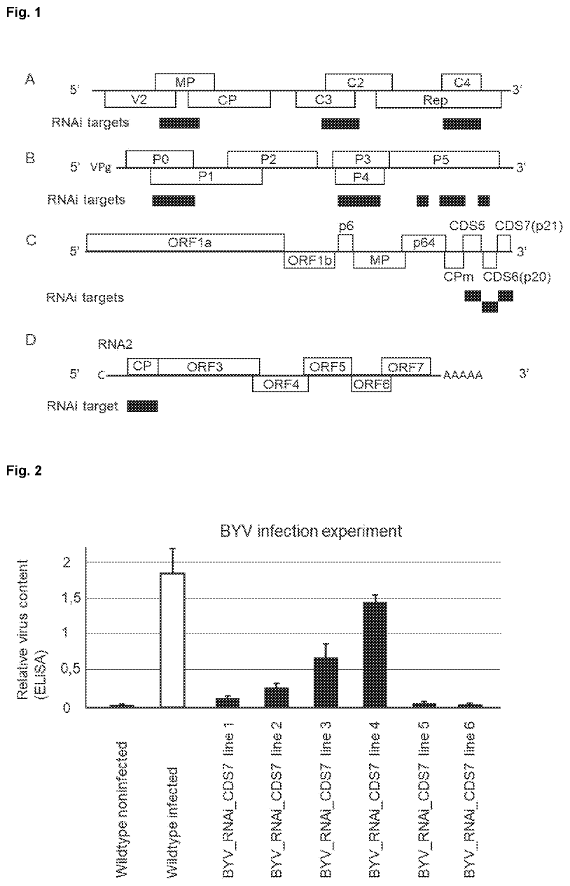

It is known that not all regions of the viral genome are suitable targets to generate high levels of virus resistance using RNA silencing mechanisms or a site-specific nuclease system (e.g. a CRISPR system) as described above. Therefore, in the context of the present invention, the most efficient target was identified for BNYVV, BCTV, BSCTV, BYV, BMYV and BWYV by analyzing stable transgenic sugar beet plants. The effect of the different RNAi constructs containing sense and antisense regions homolog to different viral target regions was analyzed in resistance assays. The tested targets can be inferred from FIG. 1. The most effective target region for each virus was selected. These target regions were combined in one genetic construct and used for the generation of transgenic sugar beet plants.

For the nuclease approach, it is essential to identify conserved regions for the design of the crRNAs since the viral genomes are relatively variable depending on the geographic region (pathotypes). To develop a durable resistance to at least two viruses, conserved regions are used to design different crRNAs for each virus. These are then combined in one DNA construct. For the identification of these conserved regions publicly available data can be used using bioinformatics tools.

In one embodiment of the nucleic acid construct or set of nucleic acid constructs described above, the nucleic acid construct or the set of nucleic acid constructs comprises or encodes at least two corresponding sense and antisense sequences, wherein the sense sequences are selected from the sequences of SEQ ID NO: 1, SEQ ID NO: 2, SEQ ID NO: 3, SEQ ID NO: 4 and SEQ ID NO: 5 or fragments thereof or sequences having a sequence identity of at least 80%, at least 85%, at least 90%, at least 95% to the sequences of SEQ ID NO: 1, SEQ ID NO: 2, SEQ ID NO: 3, SEQ ID NO: 4 and SEQ ID NO: 5 or fragments thereof.

Preferably, the fragments of the respective sequences have a length of at least 20 nt, at least 25 nt, at least 30 nt, at least 40 nt, at least 50 nt, at least 60 nt, at least 70 nt, at least 80 nt, at least 90 nt, at least 100 nt, at least 110 nt, at least 120 nt, at least 130 nt, at least 140 nt, at least 150 nt, at least 160 nt, at least 170 nt, at least 180 nt, at least 190 nt, at least 200 nt, at least 220 nt, at least 250 nt, at least 270 nt or at least 300 nt.

As explained in more detail in the examples below, for BCTV and BSCTV the overlapping C4/Rep targets (SEQ ID NOs: 3 and 4) were found to be the most effective, for BMYV, BWYV and BChV the overlapping P3/P4 target (SEQ ID NO: 2) was found to be the most effective, for BYV the CDS7 target (SEQ ID NO: 1) was found to be the most effective and for BNYVV, the CP target (SEQ ID NO: 5) was found to be the most effective, respectively.

P3 of BMYV, BWYV and BChV are orthologues as well as P4 of these viruses. Further orthologues are P3 and P4, respectively, of Potato Leaf Roll Virus (PLRV) and Cereal Yellow Dwarf Virus (CYDV).

In one embodiment of the nucleic acid construct or a set of nucleic acid constructs described above, the nucleic acid construct or the set of nucleic acid constructs comprises or encodes at least the sequences of SEQ ID NO: 3 and SEQ ID NO: 4 or fragments thereof or sequences having a sequence identity of at least 80%, at least 85%, at least 90%, at least 95% to the sequences of SEQ ID NO: 3 and SEQ ID NO: 4 or fragments thereof and the corresponding antisense sequences, or wherein the nucleic acid construct or the set of nucleic acid constructs comprises or encodes at least the sequences of SEQ ID NO: 3, SEQ ID NO: 4 and SEQ ID NO: 5 or fragments thereof or sequences having a sequence identity of at least 80%, at least 85%, at least 90%, at least 95% to the sequences of SEQ ID NO: 3, SEQ ID NO: 4 and SEQ ID NO: 5 or fragments thereof and the corresponding antisense sequences, or wherein the nucleic acid construct or the set of nucleic acid constructs comprises or encodes at least the sequences of SEQ ID NO: 1, SEQ ID NO: 2, SEQ ID NO: 3, SEQ ID NO: 4 and SEQ ID NO: 5 or fragments thereof or sequences having a sequence identity of at least 80%, at least 85%, at least 90%, at least 95% to the sequences of SEQ ID NO: 1, SEQ ID NO: 2, SEQ ID NO: 3, SEQ ID NO: 4 and SEQ ID NO: 5 or fragments thereof and the corresponding antisense sequences.

Preferably, the fragments of the respective sequences have a length of at least 20 nt, at least 25 nt, at least 30 nt, at least 40 nt, at least 50 nt, at least 60 nt, at least 70 nt, at least 80 nt, at least 90 nt, at least 100 nt, at least 110 nt, at least 120 nt, at least 130 nt, at least 140 nt, at least 150 nt, at least 160 nt, at least 170 nt, at least 180 nt, at least 190 nt, at least 200 nt, at least 220 nt, at least 250 nt, at least 270 nt or at least 300 nt.

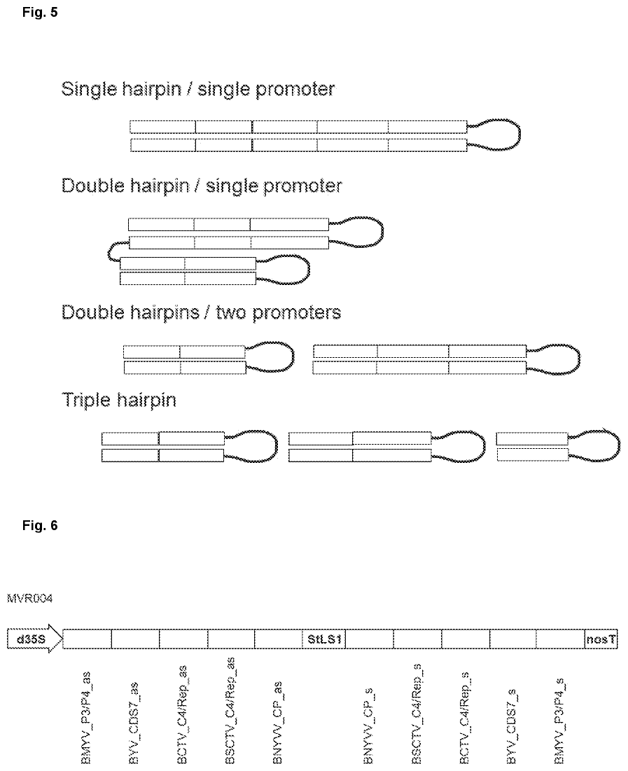

In the nucleic acid construct or set of constructs according to any of the embodiments described above, the sense and antisense sequences can be arranged to form different hairpin structures upon transcription as illustrated in FIG. 5.

In one embodiment, the nucleic acid construct or set of nucleic acid constructs according to any of the embodiments described above comprises a sense and an antisense sequence targeting a genomic sequence of each of at least two of the viruses, wherein the sense and antisense sequences are arranged in the construct:
(i) under the control of at least one promoter so that they form a single hairpin, optionally upon transcription;
(ii) under the control of at least one promoter so that they form two hairpins, optionally upon transcription;
(iii) under the control of at least two promoters so that they form two hairpins, optionally upon transcription; or
(iv) under the control of at least three promoters so that they form three hairpins, optionally upon transcription.

In this embodiment, each hairpin comprises at least one intron forming a loop between the sense and antisense sequence(s) and; in the case of a single hairpin, the hairpin is flanked by at least one terminator at the end opposite to the at least one promoter; and in case of two hairpins under the control of one promoter, the two hairpins are separated by at least one spacer; and in case of two hairpins under the control of two promoters or three hairpins under the control of three promoters, each hairpin is flanked by at least one promoter and at least one terminator.

The promoter(s) is/are preferably selected from cauliflower mosaic virus 35S promoter (d35S), Ubiquitin10 promoter from Arabidopsis (AtUbi10), Ubiquitin4 promoter from Parsley (Petroselinum crispum) (PcUbi4) and/or the intron(s) is/are selected from StLS1 and AtAAP6 and/or the terminator(s) is/are selected from nosT, oscT and 35ST.

In a preferred embodiment of the nucleic acid construct or set of nucleic acid constructs described above, the nucleic acid construct or the set of nucleic acid constructs comprises or encodes the corresponding sense and antisense sequences arranged as follows:
(a) the sense sequences targeting all of the seven viruses recited above, six of the viruses recited above, five of the viruses recited above, four of the viruses recited above, three of the viruses recited above or two of the viruses recited above are arranged in contiguous order and the corresponding antisense sequences are arranged in the reverse contiguous order, or
(b) the sense sequences targeting five and two of the viruses recited above or four and three of the viruses recited above are arranged in contiguous order, respectively, and the corresponding antisense sequences are arranged in the reverse contiguous order, or
(c) the sense sequences targeting five of the viruses recited above are arranged in contiguous order and the corresponding antisense sequences are arranged in the reverse contiguous order while the sense sequences targeting the remaining two viruses recited above and the corresponding antisense sequences are not arranged in contiguous order, or the sense sequences targeting four and two of the viruses recited above are arranged in contiguous order, respectively, and the corresponding antisense sequences are arranged in the reverse contiguous order, or the sense sequences targeting three and three of the viruses recited above are arranged in contiguous order, respectively, and the corresponding antisense sequences are arranged in the reverse contiguous order, or the sense sequences targeting three, two and two of the viruses recited above are arranged in contiguous order, respectively, and the corresponding antisense sequences are arranged in the reverse contiguous order, or
(d) the sense sequences targeting two, two and two of the viruses recited above are arranged in contiguous order, respectively, and the corresponding antisense sequences are arranged in the reverse contiguous order, or the sense sequences targeting three and two of the viruses recited above are arranged in contiguous order, respectively, and the corresponding antisense sequences are arranged in the reverse contiguous order while the sense sequences targeting the remaining two viruses recited above and the corresponding antisense sequences are not arranged in contiguous order, or the sense sequences targeting four of the viruses recited above are arranged in contiguous order and the corresponding antisense sequences are arranged in the reverse contiguous order while the sense sequences targeting the remaining three viruses recited above and the corresponding antisense sequences are not arranged in contiguous order, or
(e) the sense sequences targeting two and two of the viruses recited above are arranged in contiguous order, respectively, and the corresponding antisense sequences are arranged in the reverse contiguous order while the sense sequences targeting the remaining three viruses recited above and the corresponding antisense sequences are not arranged in contiguous order, or the sense sequences targeting three of the viruses recited above are arranged in contiguous order and the corresponding antisense sequences are arranged in the reverse contiguous order while the sense sequences targeting the remaining four viruses recited above and the corresponding antisense sequences are not arranged in contiguous order, or
(f) the sense sequences targeting two of the viruses recited above are arranged in contiguous order and the corresponding antisense sequences are arranged in the reverse contiguous order while the sense sequences targeting the remaining five viruses recited above and the corresponding antisense sequences are not arranged in contiguous order, or
(g) none of the sense sequences targeting the seven viruses recited above or the corresponding antisense sequences are arranged in contiguous order.

In the embodiment described above, preferably the sense sequences are selected from the sequences of SEQ ID NO: 1, SEQ ID NO: 2, SEQ ID NO: 3, SEQ ID NO: 4 and SEQ ID NO: 5 or fragments thereof or sequences having a sequence identity of at least 80%, at least 85%, at least 90%, at least 95% to the sequences of SEQ ID NO: 1, SEQ ID NO: 2, SEQ ID NO: 3, SEQ ID NO: 4 and SEQ ID NO: 5 or fragments thereof.

Preferably, the fragments of the respective sequences have a length of at least 20 nt, at least 25 nt, at least 30 nt, at least 40 nt, at least 50 nt, at least 60 nt, at least 70 nt, at least 80 nt, at least 90 nt, at least 100 nt, at least 110 nt, at least 120 nt, at least 130 nt, at least 140 nt, at least 150 nt, at least 160 nt, at least 170 nt, at least 180 nt, at least 190 nt, at least 200 nt, at least 220 nt, at least 250 nt, at least 270 nt or at least 300 nt.

The sense and antisense sequences can be arranged in the nucleic acid construct or set of constructs of the present invention in one, two, three, four, five, six or seven hairpins. One construct or set of construct according to the invention can comprise the sense and antisense sequences targeting all seven viruses, i.e. Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV) Beet Western Yellowing Virus (BWYV) and Beet Chlorosis Virus (BChV). In case of two hairpins targeting all seven viruses, one of the hairpins may comprise the sense and antisense sequence targeting one virus and the other hairpin comprises the sense and antisense sequences targeting the six remaining viruses, i.e. a [6+1] arrangement. Alternatively, one hairpin may comprise the sense and antisense sequences targeting five of the viruses and the other hairpin comprises the sense and antisense sequences targeting the two remaining viruses, i.e. a [5+2] arrangement. Alternatively, one hairpin may comprise the sense and antisense sequences targeting four of the viruses and the other hairpin comprises the sense and antisense sequences targeting the three remaining viruses, i.e. a [4+3] arrangement. In case of three hairpins, accordingly, [5+1+1], [4+2+1], [3+3+1] and [3+2+2] arrangements are possible; in case of four hairpins, [4+1+1+1], [3+2+1+1] and [2+2+2+1] arrangements are possible; in case of five hairpins [3+1+1+1+1] and [2+2+1+1+1] arrangements are possible; in case of six hairpins a [2+1+1+1+1+1] arrangement is possible and in case of seven hairpins a [1+1+1+1+1+1+1] arrangement is possible. One construct or set of constructs according to the invention can also comprise the sense and antisense sequences targeting six of the viruses mentioned above. In this case, for two hairpins, a [5+1], [4+2] or [3+3] arrangement is possible. For three hairpins, a [4+1+1], [3+2+1] or [2+2+2] arrangement is possible; for four hairpins, a [3+1+1+1] or [2+2+1+1] arrangement is possible; for five hairpins a [2+1+1+1+1] arrangement is possible; and for six hairpins a [1+1+1+1+1+1] arrangement is possible. One construct or set of constructs according to the invention can also comprise the sense and antisense sequences targeting five of the viruses mentioned above. In this case, for two hairpins, a [4+1] or [3+2] arrangement is possible. For three hairpins, a [3+1+1] or [2+2+1] arrangement is possible; for four hairpins, a [2+1+1+1] arrangement is possible; and for five hairpins a [1+1+1+1+1] arrangement is possible. One construct or set of constructs according to the invention can also comprise the sense and antisense sequences targeting four of the viruses mentioned above. In this case, for two hairpins, a [3+1] or [2+2] arrangement is possible. For three hairpins, a [2+1+1] arrangement is possible; and for four hairpins, a [1+1+1+1] arrangement is possible. One construct or set of constructs according to the invention can also comprise the sense and antisense sequences targeting three of the viruses mentioned above. In this case, for two hairpins, a [2+1] arrangement is possible. For three hairpins, a [1+1+1] arrangement is possible. Finally, one construct or set of constructs according to the invention can also comprise the sense and antisense sequences targeting two of the viruses mentioned above. In this case, for two hairpins, a [1+1] arrangement is possible.

In one embodiment of the nucleic acid construct or set of constructs as described in any on the embodiments above, the nucleic acid construct or set of constructs comprises or encodes corresponding sense and antisense sequences targeting

a) genomic sequences of all seven viruses recited above or orthologue sequences thereof and the sequences targeting these seven viruses are arranged in one of the following orders: i) the sense sequences targeting these seven viruses are arranged in contiguous order and the corresponding antisense sequences are arranged in the reverse contiguous order (i.e. [7]), ii) the sense sequences targeting six of the seven viruses are arranged in contiguous order and the corresponding antisense sequences are arranged in the reverse contiguous order (i.e. [6+1]), iii) the sense sequences targeting five of the seven viruses are arranged in contiguous order with the corresponding antisense sequences being arranged in the reverse contiguous order, and the sense sequences targeting the remaining two of these seven viruses are further arranged either in contiguous order or in a non-contiguous order and the corresponding antisense sequences are arranged in the reverse contiguous or non-contiguous order (i.e. [5+2] or [5+1+1]), iv) the sense sequences targeting a first subset of four of the seven viruses are arranged in contiguous order with the corresponding antisense sequences being arranged in the reverse contiguous order, and the sense sequences targeting the remaining three viruses are further either (a) arranged in contiguous order with the corresponding antisense sequences being arranged in the reverse contiguous order (i.e. [4+3]) or (b) arranged in a subset of two sense sequences targeting a subset of two of the three remaining viruses in contiguous order with the corresponding antisense sequences being arranged in the reverse contiguous order and with the sense sequence targeting the last of the six viruses and the corresponding antisense sequence not being arranged in contiguous order (i.e. [4+2+1]), or (c) arranged with the sense sequences targeting the remaining three of the seven viruses and the corresponding antisense sequences not being arranged in contiguous order (i.e. [4+1+1+1]), v) the sense sequences targeting a first subset of three of the six viruses are arranged in contiguous order with the corresponding antisense sequences being arranged in the reverse contiguous order, and (a) the sense sequences targeting three of the remaining four viruses are also arranged in contiguous order and the corresponding antisense sequences are also arranged in the reverse contiguous order and the sense sequence targeting the remaining virus and the corresponding antisense sequence are not arranged in contiguous order (i.e. [3+3+1]), or (b) the sense sequences targeting the remaining four viruses are arranged in a first subset of two sense sequences targeting a subset of two of the remaining four viruses arranged in contiguous order with the corresponding antisense sequences being arranged in the reverse contiguous order and a second subset of two sense sequences targeting the two remaining viruses of the four viruses arranged in contiguous order with the corresponding antisense sequences being arranged in the reverse contiguous order (i.e. [3+2+2]), or (c) the sense sequences targeting the remaining four viruses are arranged in a subset of two sense sequences targeting a subset of two of the remaining four viruses arranged in contiguous order with the corresponding antisense sequences arranged in the reverse contiguous order and the two sense sequences targeting the two remaining viruses of the four viruses and the corresponding antisense sequences not arranged in contiguous order (i.e. [3+2+1+1]), or where (d) the sense sequences targeting the remaining four viruses and the corresponding antisense sequences are arranged in a non-contiguous order (i.e. [3+1+1+1+1]), vi) the sense sequences targeting a first subset of two of the seven viruses are arranged in contiguous order with the corresponding antisense sequences being arranged in the reverse contiguous order, and (a) the sense sequences targeting the remaining five viruses are arranged as a first subset of two sense sequences targeting a subset of two of the remaining five viruses arranged in contiguous order with the corresponding antisense sequences being arranged in the reverse contiguous order and a second subset of two sense sequences targeting two viruses of the five remaining viruses arranged in contiguous order with the corresponding antisense sequences being arranged in the reverse contiguous order and the sense sequence targeting the remaining virus and the corresponding antisense sequence not arranged in contiguous order (i.e. [2+2+2+1]), or (b) the sense sequences targeting the remaining five viruses are arranged as a first subset of two sense sequences targeting a subset of two of the remaining five viruses arranged in contiguous order with the corresponding antisense sequences arranged in the reverse contiguous order and the three sense sequences targeting the three remaining viruses of the five viruses and the corresponding antisense sequences not arranged in contiguous order (i.e. [2+2+1+1+1]), or (c) the sense sequences targeting the remaining five viruses and the corresponding antisense sequences are arranged in a non-contiguous order (i.e. [2+1+1+1+1+1]), or vii) the sense sequences targeting these seven viruses and the corresponding antisense sequences are not arranged in contiguous order (i.e. [1+1+1+1+1+1+1]); or

b) genomic sequences of a subset of six of the seven viruses recited above or orthologue sequences thereof and the sequences targeting these six viruses are arranged in one of the following orders: i) the sense sequences targeting these six viruses are arranged in contiguous order and the corresponding antisense sequences are arranged in the reverse contiguous order (i.e. [6]), ii) the sense sequences targeting five of the six viruses are arranged in contiguous order and the corresponding antisense sequences are arranged in the reverse contiguous order (i.e. [5+1]), iii) the sense sequences targeting four of the six viruses are arranged in contiguous order with the corresponding antisense sequences arranged in the reverse contiguous order, and the sense sequences further targeting the remaining two of these six viruses are arranged either in contiguous order or in non-contiguous order and the corresponding antisense sequences are arranged in the reverse contiguous or non-contiguous order (i.e. [4+2] or [4+1+1]), iv) the sense sequences targeting a first subset of three of the six viruses are arranged in contiguous order with the corresponding antisense sequences being arranged in the reverse contiguous order, whereas the sense sequences targeting the remaining three viruses are either (a) arranged in contiguous order with the corresponding antisense sequences being arranged in the reverse contiguous order (e.g. [3+3]) or (b) arranged in a subset of two sense sequences targeting a subset of two of the three remaining viruses in contiguous order with the corresponding antisense sequences being arranged in the reverse contiguous order and with the sense sequence targeting the last of the six viruses and the corresponding antisense sequence not being arranged in contiguous order (i.e. [3+2+1]), or (c) arranged with the sense sequences targeting the remaining three of the six viruses and the corresponding antisense sequences not being arranged in contiguous order (i.e. [3+1+1+1]), v) the sense sequences targeting a first subset of two of the six viruses are arranged in contiguous order with the corresponding antisense sequences being arranged in the reverse contiguous order, whereas (a) the sense sequences targeting the remaining four viruses are arranged in a first subset of two sense sequences targeting a subset of two of the remaining four viruses arranged in contiguous order with the corresponding antisense sequences being arranged in the reverse contiguous order and a second subset of two sense sequences targeting the other two viruses of the four viruses are arranged in contiguous order with the corresponding antisense sequences being arranged in the reverse contiguous order (i.e. [2+2+2]), or where (b) the sense sequences targeting the remaining four viruses are arranged in a first subset of two sense sequences targeting a subset of two of the remaining four viruses arranged in contiguous order with the corresponding antisense sequences being arranged in the reverse contiguous order and the two sense sequences targeting the two remaining viruses of the four viruses and the corresponding antisense sequences are not arranged in contiguous order (i.e. [2+2+1+1]), or where (c) the sense sequences targeting the remaining four viruses and the corresponding antisense sequences are arranged in a non-contiguous order (i.e. [2+1+1+1+1]), or vi) the sense sequences targeting these six viruses and the corresponding antisense sequences are not arranged in contiguous order (i.e. [1+1+1+1+1+1]); or

c) genomic sequences of a subset of five of the seven viruses recited above or orthologue sequences thereof and the sequences targeting these five viruses are arranged in one of the following orders: i) the sense sequences targeting these five viruses are arranged in contiguous order and the corresponding antisense sequences are arranged in the reverse contiguous order (i.e. [5]), ii) the sense sequences targeting four of the five viruses are arranged in contiguous order and the corresponding antisense sequences are arranged in the reverse contiguous order (i.e. [4+1]), iii) the sense sequences targeting three of the five viruses are arranged in contiguous order with the corresponding antisense sequences being arranged in the reverse contiguous order, and the sense sequences further targeting the remaining two of these five viruses are arranged either in contiguous order or in non-contiguous order and the corresponding antisense sequences are arranged in the reverse contiguous order or in reverse non-contiguous order (i.e. [3+2] or [3+1+1]), iv) the sense sequences targeting two of the five viruses are arranged in contiguous order with the corresponding antisense sequences being arranged in the reverse contiguous order, and (a) the sense sequences targeting two of the remaining three viruses are also arranged in contiguous order and the corresponding antisense sequences are also arranged in the reverse contiguous order (i.e. [2+2+1]) or (b) the sense sequences targeting the remaining three viruses and the corresponding antisense sequences are not arranged in contiguous order (i.e. [2+1+1+1]), or v) the sense sequences targeting these five viruses and the corresponding antisense sequences are not arranged in contiguous order (i.e. [1+1+1+1+1]); or

d) genomic sequences of a subset of four of the seven viruses recited above or orthologue sequences thereof and the sequences targeting these four viruses are arranged in one of the following orders: i) the sense sequences targeting these four viruses are arranged in contiguous order and the corresponding antisense sequences are arranged in the reverse contiguous order (i.e. [4]), ii) the sense sequences targeting three of the four viruses are arranged in contiguous order and the corresponding antisense sequences are arranged in the reverse contiguous order (i.e. [3+1]), iii) the sense sequences targeting two of the four viruses are arranged in contiguous order with the corresponding antisense sequences being arranged in the reverse contiguous order, and the further sense sequences targeting the remaining two of these four viruses are arranged either in contiguous order with the corresponding antisense sequences arranged in the reverse contiguous order or in a non-contiguous order with the corresponding antisense sequences also not being arranged in a contiguous order (i.e. [2+2] or [2+1+1]), or iv) the sense sequences targeting these four viruses and the corresponding antisense sequences are not arranged in contiguous order (i.e. [1+1+1+1]); or

e) genomic sequences of a subset of three of the seven viruses recited above or orthologue sequences thereof and the sequences targeting these three viruses are arranged in one of the following orders: i) the sense sequences targeting these three viruses are arranged in contiguous order and the corresponding antisense sequences are arranged in the reverse contiguous order (i.e. [3]), ii) the sense sequences targeting two of the three viruses are arranged in contiguous order and the corresponding antisense sequences are arranged in the reverse contiguous order (i.e. [2+1]) or iii) the sense sequences targeting these three viruses and the corresponding antisense sequences are not arranged in contiguous order (i.e. [1+1+1]); or

f) genomic sequences of a subset of two of the seven viruses recited above or orthologue sequences thereof and the sequences targeting these two viruses are arranged in one of the following orders: i) the sense sequences targeting these two viruses are arranged in contiguous order and the corresponding antisense sequences are arranged in the reverse contiguous order (i.e. [2]), or ii) the sense sequences targeting these two viruses and the corresponding antisense sequences are not arranged in contiguous order (i.e. [1+1]).

In another embodiment of the nucleic acid construct or set of nucleic acid constructs described above, the nucleic acid construct or set of nucleic acid constructs comprises or encodes one of the following sequential arrangements:
(a) at least one promoter controlling transcription of the antisense sequence of SEQ ID NO: 2 or a fragment thereof or a sequence having a sequence identity of at least 80%, at least 85%, at least 90%, at least 95% to the antisense sequence of SEQ ID NO: 2 or a fragment thereof, the antisense sequence of SEQ ID NO: 1 or a fragment thereof or a sequence having a sequence identity of at least 80%, at least 85%, at least 90%, at least 95% to the antisense sequence of SEQ ID NO: 1 or a fragment thereof, the antisense sequence of SEQ ID NO: 3 or a fragment thereof or a sequence having a sequence identity of at least 80%, at least 85%, at least 90%, at least 95% to the antisense sequence of SEQ ID NO: 3 or a fragment thereof, the antisense sequence of SEQ ID NO: 4 or a fragment thereof or a sequence having a sequence identity of at least 80%, at least 85%, at least 90%, at least 95% to the antisense sequence of SEQ ID NO: 4 or a fragment thereof, the antisense sequence of SEQ ID NO: 5 or a fragment thereof or a sequence having a sequence identity of at least 80%, at least 85%, at least 90%, at least 95% to the antisense sequence of SEQ ID NO: 5 or a fragment thereof, at least one intron, the sequence of SEQ ID NO: 5 or a fragment thereof or a sequence having a sequence identity of at least 80%, at least 85%, at least 90%, at least 95% to the sequence of SEQ ID NO: 5 or a fragment thereof, the sequence of SEQ ID NO: 4 or a fragment thereof or a sequence having a sequence identity of at least 80%, at least 85%, at least 90%, at least 95% to the sequence of SEQ ID NO: 4 or a fragment thereof, the sequence of SEQ ID NO: 3 or a fragment thereof or a sequence having a sequence identity of at least 80%, at least 85%, at least 90%, at least 95% to the sequence of SEQ ID NO: 3 or a fragment thereof, the sequence of SEQ ID NO: 1 or a fragment thereof or a sequence having a sequence identity of at least 80%, at least 85%, at least 90%, at least 95% to the sequence of SEQ ID NO: 1 or a fragment thereof, the sequence of SEQ ID NO: 2 or a fragment thereof or a sequence having a sequence identity of at least 80%, at least 85%, at least 90%, at least 95% to the sequence of SEQ ID NO: 2 or a fragment thereof, at least one terminator terminating the transcription of the former sequences;
(b) at least one promoter controlling transcription of the sequence of SEQ ID NO: 2 or a fragment thereof or a sequence having a sequence identity of at least 80%, at least 85%, at least 90%, at least 95% to the sequence of SEQ ID NO: 2 or a fragment thereof, the sequence of SEQ ID NO: 1 or a sequence having a sequence identity of at least 80%, at least 85%, at least 90%, at least 95% to the sequence of SEQ ID NO: 1 or a fragment thereof, the sequence of SEQ ID NO: 3 or a fragment thereof or a sequence having a sequence identity of at least 80%, at least 85%, at least 90%, at least 95% to the sequence of SEQ ID NO: 3 or a fragment thereof, the sequence of SEQ ID NO: 4 or a fragment thereof or a sequence having a sequence identity of at least 80%, at least 85%, at least 90%, at least 95% to the sequence of SEQ ID NO: 4 or a fragment thereof, the sequence of SEQ ID NO: 5 or a fragment thereof or a sequence having a sequence identity of at least 80%, at least 85%, at least 90%, at least 95% to the sequence of SEQ ID NO: 5 or a fragment thereof, at least one intron, the antisense sequence of SEQ ID NO: 5 or a fragment thereof or a sequence having a sequence identity of at least 80%, at least 85%, at least 90%, at least 95% to the antisense sequence of SEQ ID NO: 5 or a fragment thereof, the antisense sequence of SEQ ID NO: 4 or a sequence having a sequence identity of at least 80%, at least 85%, at least 90%, at least 95% to the antisense sequence of SEQ ID NO: 4 or a fragment thereof, the antisense sequence of SEQ ID NO: 3 or a fragment thereof or a sequence having a sequence identity of at least 80%, at least 85%, at least 90%, at least 95% to the antisense sequence of SEQ ID NO: 3 or a fragment thereof, the antisense sequence of SEQ ID NO: 1 or a fragment thereof or a sequence having a sequence identity of at least 80%, at least 85%, at least 90%, at least 95% to the antisense sequence of SEQ ID NO: 1 or a fragment thereof, the antisense sequence of SEQ ID NO: 2 or a fragment thereof or a sequence having a sequence identity of at least 80%, at least 85%, at least 90%, at least 95% to the antisense sequence of SEQ ID NO: 2 or a fragment thereof, at least one terminator terminating the transcription of the former sequences;
(c) at least one promoter controlling the transcription of the antisense sequence of SEQ ID NO: 2 or a fragment thereof or a sequence having a sequence identity of at least 80%, at least 85%, at least 90%, at least 95% to the antisense sequence of SEQ ID NO: 2 or a fragment thereof, the antisense sequence of SEQ ID NO: 1 or a fragment thereof or a sequence having a sequence identity of at least 80%, at least 85%, at least 90%, at least 95% to the antisense sequence of SEQ ID NO: 1 or a fragment thereof, the antisense sequence of SEQ ID NO: 5 or a fragment thereof or a sequence having a sequence identity of at least 80%, at least 85%, at least 90%, at least 95% to the antisense sequence of SEQ ID NO: 5 or a fragment thereof, at least one intron, the sequence of SEQ ID NO: 5 or a fragment thereof or a sequence having a sequence identity of at least 80%, at least 85%, at least 90%, at least 95% to the sequence of SEQ ID NO: 5 or a fragment thereof, the sequence of SEQ ID NO: 1 or a fragment thereof or a sequence having a sequence identity of at least 80%, at least 85%, at least 90%, at least 95% to the sequence of SEQ ID NO: 1 or a fragment thereof, the sequence of SEQ ID NO: 2 or a fragment thereof or a sequence having a sequence identity of at least 80%, at least 85%, at least 90%, at least 95% to the sequence of SEQ ID NO: 2 or a fragment thereof, at least one spacer, the antisense sequence of SEQ ID NO: 3 or a fragment thereof or a sequence having a sequence identity of at least 80%, at least 85%, at least 90%, at least 95% to the antisense sequence of SEQ ID NO: 3 or a fragment thereof, the antisense sequence of SEQ ID NO: 4 or a fragment thereof or a sequence having a sequence identity of at least 80%, at least 85%, at least 90%, at least 95% to the antisense sequence of SEQ ID NO: 4 or a fragment thereof, at least one intron, the sequence of SEQ ID NO: 4 or a fragment thereof or a sequence having a sequence identity of at least 80%, at least 85%, at least 90%, at least 95% to the sequence of SEQ ID NO: 4 or a fragment thereof, the sequence of SEQ ID NO: 3 or a fragment thereof or a sequence having a sequence identity of at least 80%, at least 85%, at least 90%, at least 95% to the sequence of SEQ ID NO: 3 or a fragment thereof, at least one terminator terminating the transcription of the former sequences;
(d) at least one terminator terminating the transcription of the sequence of SEQ ID NO: 1 or a fragment thereof or a sequence having a sequence identity of at least 80%, at least 85%, at least 90%, at least 95% to the sequence of SEQ ID NO: 1 or a fragment thereof, the sequence of SEQ ID NO: 2 or a fragment thereof or a sequence having a sequence identity of at least 80%, at least 85%, at least 90%, at least 95% to the sequence of SEQ ID NO: 2 or a fragment thereof, at least one intron, the antisense sequence of SEQ ID NO: 2 or a fragment thereof or a sequence having a sequence identity of at least 80%, at least 85%, at least 90%, at least 95% to the antisense sequence of SEQ ID NO: 2 or a fragment thereof, the antisense sequence of SEQ ID NO: 1 or a fragment thereof or a sequence having a sequence identity of at least 80%, at least 85%, at least 90%, at least 95% to the antisense sequence of SEQ ID NO: 1 or a fragment thereof, at least one promoter controlling the transcription of the former sequences, at least one promoter controlling the transcription of the antisense sequence of SEQ ID NO: 3 or a fragment thereof or a sequence having a sequence identity of at least 80%, at least 85%, at least 90%, at least 95% to the antisense sequence of SEQ ID NO: 3 or a fragment thereof, the antisense sequence of SEQ ID NO: 4 or a fragment thereof or a sequence having a sequence identity of at least 80%, at least 85%, at least 90%, at least 95% to the antisense sequence of SEQ ID NO: 4 or a fragment thereof, the antisense sequence of SEQ ID NO: 5 or a fragment thereof or a sequence having a sequence identity of at least 80%, at least 85%, at least 90%, at least 95% to the antisense sequence of SEQ ID NO: 5 or a fragment thereof, at least one intron, the sequence of SEQ ID NO: 5 or a fragment thereof or a sequence having a sequence identity of at least 80%, at least 85%, at least 90%, at least 95% to the sequence of SEQ ID NO: 5 or a fragment thereof, the sequence of SEQ ID NO: 4 or a fragment thereof or a sequence having a sequence identity of at least 80%, at least 85%, at least 90%, at least 95% to the sequence of SEQ ID NO: 4 or a fragment thereof, the sequence of SEQ ID NO: 3 or a fragment thereof or a sequence having a sequence identity of at least 80%, at least 85%, at least 90%, at least 95% to the sequence of SEQ ID NO: 3 or a fragment thereof, at least one terminator terminating the transcription of the former sequences;
(e) at least one promoter controlling the transcription of the antisense sequence of SEQ ID NO: 5 or a fragment thereof or a sequence having a sequence identity of at least 80%, at least 85%, at least 90%, at least 95% to the antisense sequence of SEQ ID NO: 5 or a fragment thereof, the antisense sequence of SEQ ID NO: 2 or a fragment thereof or a sequence having a sequence identity of at least 80%, at least 85%, at least 90%, at least 95% to the antisense sequence of SEQ ID NO: 2 or a fragment thereof, the antisense sequence of SEQ ID NO: 1 or a fragment thereof or a sequence having a sequence identity of at least 80%, at least 85%, at least 90%, at least 95% to the antisense sequence of SEQ ID NO: 1 or a fragment thereof, at least one intron, the sequence of SEQ ID NO: 1 or a fragment thereof or a sequence having a sequence identity of at least 80%, at least 85%, at least 90%, at least 95% to the sequence of SEQ ID NO: 1 or a fragment thereof, the sequence of SEQ ID NO: 2 or a fragment thereof or a sequence having a sequence identity of at least 80%, at least 85%, at least 90%, at least 95% to the sequence of SEQ ID NO: 2 or a fragment thereof, the sequence of SEQ ID NO: 5 or a fragment thereof or a sequence having a sequence identity of at least 80%, at least 85%, at least 90%, at least 95% to the sequence of SEQ ID NO: 5 or a fragment thereof, at least one terminator terminating the transcription of the former sequences, at least one terminator terminating the transcription of the antisense sequence of SEQ ID NO: 3 or a fragment thereof or a sequence having a sequence identity of at least 80%, at least 85%, at least 90%, at least 95% to the antisense sequence of SEQ ID NO: 3 or a fragment thereof, the antisense sequence of SEQ ID NO: 4 or a fragment thereof or a sequence having a sequence identity of at least 80%, at least 85%, at least 90%, at least 95% to the antisense sequence of SEQ ID NO: 4 or a fragment thereof, at least one intron, the sequence of SEQ ID NO: 4 or a fragment thereof or a sequence having a sequence identity of at least 80%, at least 85%, at least 90%, at least 95% to the sequence of SEQ ID NO: 4 or a fragment thereof, the sequence of SEQ ID NO: 3 or a fragment thereof or a sequence having a sequence identity of at least 80%, at least 85%, at least 90%, at least 95% to the sequence of SEQ ID NO: 3 or a fragment thereof, at least one promoter controlling the transcription of the former sequences;
(f) at least one terminator terminating transcription of the sequence of SEQ ID NO: 1 or a fragment thereof or a sequence having a sequence identity of at least 80%, at least 85%, at least 90%, at least 95% to the sequence of SEQ ID NO: 1 or a fragment thereof, the sequence of SEQ ID NO: 2 or a fragment thereof or a sequence having a sequence identity of at least 80%, at least 85%, at least 90%, at least 95% to the sequence of SEQ ID NO: 2 or a fragment thereof, at least one intron, the antisense sequence of SEQ ID NO: 2 or a fragment thereof or a sequence having a sequence identity of at least 80%, at least 85%, at least 90%, at least 95% to the antisense sequence of SEQ ID NO: 2 or a fragment thereof, the antisense sequence of SEQ ID NO: 1 or a fragment thereof or a sequence having a sequence identity of at least 80%, at least 85%, at least 90%, at least 95% to the antisense sequence of SEQ ID NO: 1 or a fragment thereof, at least one promoter controlling the transcription of the former sequences, at least one promoter controlling the transcription of the antisense sequence of SEQ ID NO: 3 or a fragment thereof or a sequence having a sequence identity of at least 80%, at least 85%, at least 90%, at least 95% to the antisense sequence of SEQ ID NO: 3 or a fragment thereof, the antisense sequence of SEQ ID NO: 5 or a fragment thereof or a sequence having a sequence identity of at least 80%, at least 85%, at least 90%, at least 95% to the antisense sequence of SEQ ID NO: 5 or a fragment thereof, at least one intron, the sequence of SEQ ID NO: 5 or a sequence having a sequence identity of at least 80%, at least 85%, at least 90%, at least 95% to the sequence of SEQ ID NO: 5 or a fragment thereof, the sequence of SEQ ID NO: 3 or a fragment thereof or a sequence having a sequence identity of at least 80%, at least 85%, at least 90%, at least 95% to the sequence of SEQ ID NO: 3 or a fragment thereof, at least one terminator terminating the transcription of the former sequences, at least one promoter controlling the transcription of the antisense sequence of SEQ ID NO: 4 or a fragment thereof or a sequence having a sequence identity of at least 80%, at least 85%, at least 90%, at least 95% to the antisense sequence o SEQ ID NO: 4 or a fragment thereof, at least one intron, the sequence of SEQ ID NO: 4 or a fragment thereof or a sequence having a sequence identity of at least 80%, at least 85%, at least 90%, at least 95% to the sequence of SEQ ID NO: 4 or a fragment thereof, at least one terminator terminating the transcription of the former sequences.

Preferably, the fragments of the respective sequences have a length of at least 20 nt, at least 25 nt, at least 30 nt, at least 40 nt, at least 50 nt, at least 60 nt, at least 70 nt, at least 80 nt, at least 90 nt, at least 100 nt, at least 110 nt, at least 120 nt, at least 130 nt, at least 140 nt, at least 150 nt, at least 160 nt, at least 170 nt, at least 180 nt, at least 190 nt, at least 200 nt, at least 220 nt, at least 250 nt, at least 270 nt or at least 300 nt.

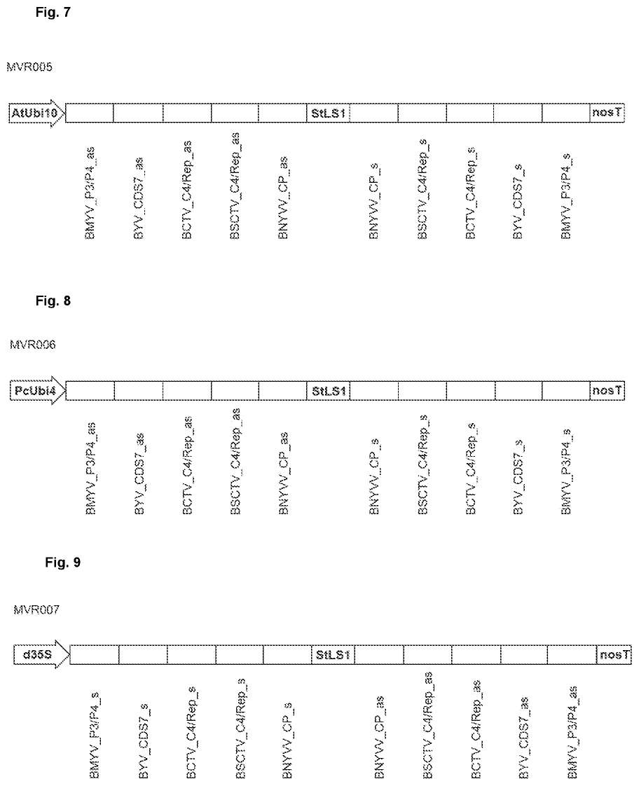

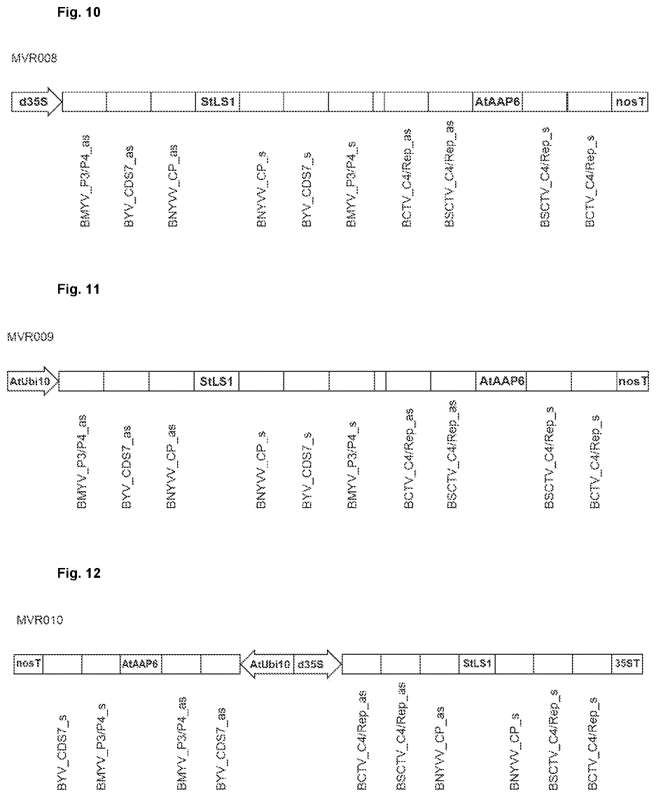

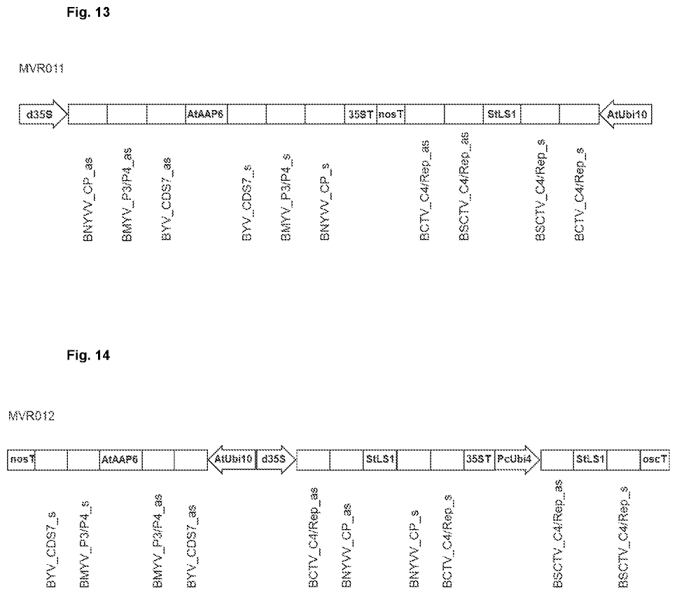

The sequential arrangement (a) as described above is shown schematically in FIGS. 6, 7 and 8, the sequential arrangement (b) is shown schematically in FIG. 9, the sequential arrangement (c) is shown schematically in FIGS. 10 and 11, the sequential arrangement (d) is shown schematically in FIG. 12, the sequential arrangement (e) is shown schematically in FIG. 13 and the sequential arrangement (f) is shown schematically in FIG. 14, respectively.

In the above described embodiments, preferably the promoter(s) is/are selected from cauliflower mosaic virus 35S promoter (d35S), Ubiquitin10 promoter from Arabidopsis (AtUbi10), Ubiquitin4 promoter from Parsley (Petroselinum crispum) (PcUbi4) and/or the intron(s) is/are selected from StLS1 and AtAAP6 and/or the terminator(s) is/are selected from nosT, oscT and 35ST.

Further preferred is an embodiment of the nucleic acid construct or set of nucleic acid constructs described above, wherein the nucleic acid construct or set of nucleic acid constructs comprises or encodes one of the following sequential arrangements:
(a) a d35S or a AtUbi10 or a PcUbi10 promoter controlling transcription of the antisense sequence of SEQ ID NO: 2 or a fragment thereof or a sequence having a sequence identity of at least 80%, at least 85%, at least 90%, at least 95% to the antisense sequence of SEQ ID NO: 2 or a fragment thereof, the antisense sequence of SEQ ID NO: 1 or a fragment thereof or a sequence having a sequence identity of at least 80%, at least 85%, at least 90%, at least 95% to the antisense sequence of SEQ ID NO: 1 or a fragment thereof, the antisense sequence of SEQ ID NO: 3 or a fragment thereof or a sequence having a sequence identity of at least 80%, at least 85%, at least 90%, at least 95% to the antisense sequence of SEQ ID NO: 3 or a fragment thereof, the antisense sequence of SEQ ID NO: 4 or a fragment thereof or a sequence having a sequence identity of at least 80%, at least 85%, at least 90%, at least 95% to the antisense sequence of SEQ ID NO: 4 or a fragment thereof, the antisense sequence of SEQ ID NO: 5 or a fragment thereof or a sequence having a sequence identity of at least 80%, at least 85%, at least 90%, at least 95% to the antisense sequence of SEQ ID NO: 5 or a fragment thereof, a StLS1 intron, the sequence of SEQ ID NO: 5 or a fragment thereof or a sequence having a sequence identity of at least 80%, at least 85%, at least 90%, at least 95% to the sequence of SEQ ID NO: 5 or a fragment thereof, the sequence of SEQ ID NO: 4 or a fragment thereof or a sequence having a sequence identity of at least 80%, at least 85%, at least 90%, at least 95% to the sequence of SEQ ID NO: 4 or a fragment thereof, the sequence of SEQ ID NO: 3 or a fragment thereof or a sequence having a sequence identity of at least 80%, at least 85%, at least 90%, at least 95% to the sequence of SEQ ID NO: 3 or a fragment thereof, the sequence of SEQ ID NO: 1 or a fragment thereof or a sequence having a sequence identity of at least 80%, at least 85%, at least 90%, at least 95% to the sequence of SEQ ID NO: 1 or a fragment thereof, the sequence of SEQ ID NO: 2 or a fragment thereof or a sequence having a sequence identity of at least 80%, at least 85%, at least 90%, at least 95% to the sequence of SEQ ID NO: 2 or a fragment thereof, a nosT terminator terminating the transcription of the former sequences;
(b) a d35S promoter controlling transcription of the sequence of SEQ ID NO: 2 or a fragment thereof or a sequence having a sequence identity of at least 80%, at least 85%, at least 90%, at least 95% to the sequence of SEQ ID NO: 2 or a fragment thereof, the sequence of SEQ ID NO: 1 or a fragment thereof or a sequence having a sequence identity of at least 80%, at least 85%, at least 90%, at least 95% to the sequence of SEQ ID NO: 1 or a fragment thereof, the sequence of SEQ ID NO: 3 or a fragment thereof or a sequence having a sequence identity of at least 80%, at least 85%, at least 90%, at least 95% to the sequence of SEQ ID NO: 3 or a fragment thereof, the sequence of SEQ ID NO: 4 or a fragment thereof or a sequence having a sequence identity of at least 80%, at least 85%, at least 90%, at least 95% to the sequence of SEQ ID NO: 4 or a fragment thereof, the sequence of SEQ ID NO: 5 or a fragment thereof or a sequence having a sequence identity of at least 80%, at least 85%, at least 90%, at least 95% to the sequence of SEQ ID NO: 5 or a fragment thereof, a StLS1 intron, the antisense sequence of SEQ ID NO: 5 or a fragment thereof or a sequence having a sequence identity of at least 80%, at least 85%, at least 90%, at least 95% to the antisense sequence of SEQ ID NO: 5 or a fragment thereof, the antisense sequence of SEQ ID NO: 4 or a fragment thereof or a sequence having a sequence identity of at least 80%, at least 85%, at least 90%, at least 95% to the antisense sequence of SEQ ID NO: 4 or a fragment thereof, the antisense sequence of SEQ ID NO: 3 or a fragment thereof or a sequence having a sequence identity of at least 80%, at least 85%, at least 90%, at least 95% to the antisense sequence of SEQ ID NO: 3 or a fragment thereof, the antisense sequence of SEQ ID NO: 1 or a fragment thereof or a sequence having a sequence identity of at least 80%, at least 85%, at least 90%, at least 95% to the antisense sequence of SEQ ID NO: 1 or a fragment thereof, the antisense sequence of SEQ ID NO: 2 or a fragment thereof or a sequence having a sequence identity of at least 80%, at least 85%, at least 90%, at least 95% to the antisense sequence of SEQ ID NO: 2 or a fragment thereof, a nosT terminator terminating the transcription of the former sequences;
(c) a d35S or a AtUbi10 promoter controlling the transcription of the antisense sequence of SEQ ID NO: 2 or a fragment thereof or a sequence having a sequence identity of at least 80%, at least 85%, at least 90%, at least 95% to the antisense sequence of SEQ ID NO: 2 or a fragment thereof, the antisense sequence of SEQ ID NO: 1 or a fragment thereof or a sequence having a sequence identity of at least 80%, at least 85%, at least 90%, at least 95% to the antisense sequence of SEQ ID NO: 1 or a fragment thereof, the antisense sequence of SEQ ID NO: 5 or a fragment thereof or a sequence having a sequence identity of at least 80%, at least 85%, at least 90%, at least 95% to the antisense sequence of SEQ ID NO: 5 or a fragment thereof, a StLS1 intron, the sequence of SEQ ID NO: 5 or a fragment thereof or a sequence having a sequence identity of at least 80%, at least 85%, at least 90%, at least 95% to the sequence of SEQ ID NO: 5 or a fragment thereof, the sequence of SEQ ID NO: 1 or a fragment thereof or a sequence having a sequence identity of at least 80%, at least 85%, at least 90%, at least 95% to the sequence of SEQ ID NO: 1 or a fragment thereof, the sequence of SEQ ID NO: 2 or a fragment thereof or a sequence having a sequence identity of at least 80%, at least 85%, at least 90%, at least 95% to the sequence of SEQ ID NO: 2 or a fragment thereof, at least one spacer, the antisense sequence of SEQ ID NO: 3 or a fragment thereof or a sequence having a sequence identity of at least 80%, at least 85%, at least 90%, at least 95% to the antisense sequence of SEQ ID NO: 3 or a fragment thereof, the antisense sequence of SEQ ID NO: 4 or a fragment thereof or a sequence having a sequence identity of at least 80%, at least 85%, at least 90%, at least 95% to the antisense sequence of SEQ ID NO: 4 or a fragment thereof, a AtAAP6 intron, the sequence of SEQ ID NO: 4 or a fragment thereof or a sequence having a sequence identity of at least 80%, at least 85%, at least 90%, at least 95% to the sequence of SEQ ID NO: 4 or a fragment thereof, the sequence of SEQ ID NO: 3 or a fragment thereof or a sequence having a sequence identity of at least 80%, at least 85%, at least 90%, at least 95% to the sequence of SEQ ID NO: 3 or a fragment thereof, a nosT terminator terminating the transcription of the former sequences;
(d) a nosT terminator terminating the transcription of the sequence of SEQ ID NO:

1 or a fragment thereof or a sequence having a sequence identity of at least 80%, at least 85%, at least 90%, at least 95% to the sequence of SEQ ID NO: 1 or a fragment thereof, the sequence of SEQ ID NO: 2 or a fragment thereof or a sequence having a sequence identity of at least 80%, at least 85%, at least 90%, at least 95% to the sequence of SEQ ID NO: 2 or a fragment thereof, a AtAAP6 intron, the antisense sequence of SEQ ID NO: 2 or a fragment thereof or a sequence having a sequence identity of at least 80%, at least 85%, at least 90%, at least 95% to the antisense sequence of SEQ ID NO: 2 or a fragment thereof, the antisense sequence of SEQ ID NO: 1 or a fragment thereof or a sequence having a sequence identity of at least 80%, at least 85%, at least 90%, at least 95% to the antisense sequence of SEQ ID NO: 1 or a fragment thereof, a AtUbi10 promoter controlling the transcription of the former sequences, a d35S promoter controlling the transcription of the antisense sequence of SEQ ID NO: 3 or a fragment thereof or a sequence having a sequence identity of at least 80%, at least 85%, at least 90%, at least 95% to the antisense sequence of SEQ ID NO: 3 or a fragment thereof, the antisense sequence of SEQ ID NO: 4 or a fragment thereof or a sequence having a sequence identity of at least 80%, at least 85%, at least 90%, at least 95% to the antisense sequence of SEQ ID NO: 4 or a fragment thereof, the antisense sequence of SEQ ID NO: 5 or a fragment thereof or a sequence having a sequence identity of at least 80%, at least 85%, at least 90%, at least 95% to the antisense sequence of SEQ ID NO: 5 or a fragment thereof, a StLS intron, the sequence of SEQ ID NO: 5 or a fragment thereof or a sequence having a sequence identity of at least 80%, at least 85%, at least 90%, at least 95% to the sequence of SEQ ID NO: 5 or a fragment thereof, the sequence of

SEQ ID NO: 4 or a fragment thereof or a sequence having a sequence identity of at least 80%, at least 85%, at least 90%, at least 95% to the sequence of SEQ ID NO: 4 or a fragment thereof, the sequence of SEQ ID NO: 3 or a fragment thereof or a sequence having a sequence identity of at least 80%, at least 85%, at least 90%, at least 95% to the sequence of SEQ ID NO: 3 or a fragment thereof, a 35ST terminator terminating the transcription of the former sequences;
(e) a d35S promoter controlling the transcription of the antisense sequence of SEQ ID NO: 5 or a fragment thereof or a sequence having a sequence identity of at least 80%, at least 85%, at least 90%, at least 95% to the antisense sequence of SEQ ID NO: 5 or a fragment thereof, the antisense sequence of SEQ ID NO: 2 or a fragment thereof or a sequence having a sequence identity of at least 80%, at least 85%, at least 90%, at least 95% to the antisense sequence of SEQ ID NO: 2 or a fragment thereof, the antisense sequence of SEQ ID NO: 1 or a fragment thereof or a sequence having a sequence identity of at least 80%, at least 85%, at least 90%, at least 95% to the antisense sequence of SEQ ID NO: 1 or a fragment thereof, a AtAAP6 intron, the sequence of SEQ ID NO: 1 or a fragment thereof or a sequence having a sequence identity of at least 80%, at least 85%, at least 90%, at least 95% to the sequence of SEQ ID NO: 1 or a fragment thereof, the sequence of SEQ ID NO: 2 or a fragment thereof or a sequence having a sequence identity of at least 80%, at least 85%, at least 90%, at least 95% to the sequence of SEQ ID NO: 2 or a fragment thereof, the sequence of SEQ ID NO: 5 or a fragment thereof or a sequence having a sequence identity of at least 80%, at least 85%, at least 90%, at least 95% to the sequence of SEQ ID NO: 5 or a fragment thereof, a 35ST terminator terminating the transcription of the former sequences, a nosT terminator terminating the transcription of the antisense sequence of SEQ ID NO: 3 or a fragment thereof or a sequence having a sequence identity of at least 80%, at least 85%, at least 90%, at least 95% to the antisense sequence of SEQ ID NO: 3 or a fragment thereof, the antisense sequence of SEQ ID NO: 4 or a fragment thereof or a sequence having a sequence identity of at least 80%, at least 85%, at least 90%, at least 95% to the antisense sequence of SEQ ID NO: 4 or a fragment thereof, a StLS1 intron, the sequence of SEQ ID NO: 4 or a fragment thereof or a sequence having a sequence identity of at least 80%, at least 85%, at least 90%, at least 95% to the sequence of SEQ ID NO: 4 or a fragment thereof, the sequence of SEQ ID NO: 3 or a fragment thereof or a sequence having a sequence identity of at least 80%, at least 85%, at least 90%, at least 95% to the sequence of SEQ ID NO: 3 or a fragment thereof, a AtUbi10 promoter controlling the transcription of the former sequences;
(f) a nosT terminator terminating transcription of the sequence of SEQ ID NO: 1 or a fragment thereof or a sequence having a sequence identity of at least 80%, at least 85%, at least 90%, at least 95% to the sequence of SEQ ID NO: 1 or a fragment thereof, the sequence of SEQ ID NO: 2 or a fragment thereof or a sequence having a sequence identity of at least 80%, at least 85%, at least 90%, at least 95% to the sequence of SEQ ID NO: 2 or a fragment thereof, a AtAAP6 intron, the antisense sequence of SEQ ID NO: 2 or a fragment thereof or a sequence having a sequence identity of at least 80%, at least 85%, at least 90%, at least 95% to the antisense sequence of SEQ ID NO: 2 or a fragment thereof, the antisense sequence of SEQ ID NO: 1 or a fragment thereof or a sequence having a sequence identity of at least 80%, at least 85%, at least 90%, at least 95% to the antisense sequence of SEQ ID NO: 1 or a fragment thereof, a AtUbi10 promoter controlling the transcription of the former sequences, a d35S promoter controlling the transcription of the antisense sequence of SEQ ID NO: 3 or a fragment thereof or a sequence having a sequence identity of at least 80%, at least 85%, at least 90%, at least 95% to the antisense sequence of SEQ ID NO: 3 or a fragment thereof, the antisense sequence of SEQ ID NO: 5 or a fragment thereof or a sequence having a sequence identity of at least 80%, at least 85%, at least 90%, at least 95% to the antisense sequence of SEQ ID NO: 5 or a fragment thereof, a StLS1 intron, the sequence of SEQ ID NO: 5 or a fragment thereof or a sequence having a sequence identity of at least 80%, at least 85%, at least 90%, at least 95% to the sequence of SEQ ID NO: 5 or a fragment thereof, the sequence of SEQ ID NO: 3 or a sequence having a sequence identity of at least 80%, at least 85%, at least 90%, at least 95% to the sequence of SEQ ID NO: 3 or a fragment thereof, a 35ST terminator terminating the transcription of the former sequences, a PcUbi4 promoter controlling the transcription of the antisense sequence of SEQ ID NO: 4 or a fragment thereof or a sequence having a sequence identity of at least 80%, at least 85%, at least 90%, at least 95% to the antisense sequence of SEQ ID NO: 4 or a fragment thereof, a StLS1 intron, the sequence of SEQ ID NO: 4 or a fragment thereof or a sequence having a sequence identity of at least 80%, at least 85%, at least 90%, at least 95% to the sequence of SEQ ID NO: 4 or a fragment thereof, a oscT terminator terminating the transcription of the former sequences.

Preferably, the fragments of the respective sequences have a length of at least 20 nt, at least 25 nt, at least 30 nt, at least 40 nt, at least 50 nt, at least 60 nt, at least 70 nt, at least 80 nt, at least 90 nt, at least 100 nt, at least 110 nt, at least 120 nt, at least 130 nt, at least 140 nt, at least 150 nt, at least 160 nt, at least 170 nt, at least 180 nt, at least 190 nt, at least 200 nt, at least 220 nt, at least 250 nt, at least 270 nt or at least 300 nt.

In one aspect, the present invention relates to an RNA molecule or a set of RNA molecules, which is/are formed upon transcription of a nucleic acid construct or set of nucleic acid constructs according to any of the embodiments described above.

When the nucleic acid construct or set of constructs according to any of the embodiments described above is transcribed in a cell into RNA, it can form different hairpin structures as described above and as e.g. illustrated in FIG. 5.

In one embodiment, the RNA molecule or set of RNA molecules described above forms one hairpin, two hairpins, three hairpins, four hairpins, five hairpins, six hairpins or seven hairpins, preferably wherein each hairpin comprises at least one intron forming a loop between the sense and antisense sequence(s) and any two hairpins are separated by at least one spacer or are present on different RNA molecules.

In another aspect, the present invention relates to a vector or a set of vectors comprising a nucleic acid construct or set of nucleic acid constructs according to any of the embodiments described above.

The nucleic acid construct or set of nucleic acid constructs of the present invention can be delivered into a Beta plant cell using a vector or set of vectors, which comprise or encode the sense and/or antisense sequences and, optionally a nucleic acid guided nuclease, as well as required regulatory elements such as promoters and terminators.

In a further aspect, the present invention provides a virus resistant transgenic Beta plant, cell of a Beta plant, part of a Beta plant or seed of a Beta plant comprising a nucleic acid construct or set of nucleic acid constructs according to any of the embodiments described above, or comprising an RNA molecule or set of RNA molecules as described above or comprising a vector or set of vectors as described above.

Preferably the Beta plant is a Beta vulgaris spp., in particular a Beta vulgaris subsp. vulgaris. The Beta plant is resistant to two viruses, three viruses, four viruses, five viruses, six viruses or all of the viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV).

In a preferred embodiment of the transgenic Beta plant described above, the nucleic acid construct or set of nucleic acid constructs according to any of the embodiments described above is chromosomally integrated into the genome of the Beta plant. Thus, the resistance is stably inherited by the offspring of the Beta plant.

To obtain such plants, integration into elite germplasm (by means of transformation or by directed insertion via crNA and endonuclease) followed by a few or even no crossing steps or integration into non-elite germplasm (by means of transformation or by directed insertion via crNA and endonuclease) followed by introgression into elite germplasm (requires more crossing steps) are possible. The second alternative represents a preferred embodiment of the present invention.

Notably, the virus resistance conferred by the construct(s) of the present invention can be combined with other desirable traits. This can be achieved by molecular stacking or via a breeding stack. Preferred desirable traits are selected from the group consisting of resistance or tolerance to abiotic stress, including drought stress, osmotic stress, heat stress, cold stress, oxidative stress, heavy metal stress, nitrogen deficiency, phosphate deficiency, salt stress or waterlogging, herbicide resistance, including resistance to glyphosate, glufosinate/phosphinotricin, hygromycin, resistance or tolerance to 2,4-D, protoporphyrinogen oxidase (PPO) inhibitors, ALS inhibitors, HPPD inhibitors and Dicamba, resistance or tolerance to biotic stress, including fungal resistance, bacterial resistance, insect resistance, or a yield related trait, including flowering time or sugar yield.

In yet another aspect, the present invention relates to a method of conferring resistance to a Beta plant to at least two viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV) comprising the steps:
(i) transforming at least one Beta plant cell with a nucleic acid construct or set of nucleic acid constructs according to any of the embodiments described above or a vector or set of vectors as described above, and
(ii) regenerating a Beta plant from the transformed cell of step (i),
(iii) optionally, selecting and/or obtaining a Beta plant, which is resistant to at least two viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV) Beet Western Yellowing Virus (BWYV) and Beet Chlorosis Virus (BChV).

Preferably the Beta plant (cell) is a Beta vulgaris spp. (cell), in particular a Beta vulgaris subsp. vulgaris (cell). More preferred, the Beta plant cell into which a nucleic acid construct or set of nucleic acid constructs according to any of the embodiments described above or a vector or set of vectors as described above is transformed is the sugar beet genotype 5706.

When a Beta cell is transformed with a nucleic acid construct or set of nucleic acid constructs according to the invention, the construct or set of constructs can persist extrachromosomally, i.e. non integrated into the genome of the target cell. Preferably, the construct is stably integrated into the genome of the target cell, including the nuclear genome or further genetic elements of the target cell, such as the genome of plastids like mitochondria or chloroplasts. After transformation, the construct or set of constructs is expressed, i.e. transcribed into RNA and/or translated. If the construct or set of constructs comprises or encodes corresponding sense and antisense sequences, the transcribed RNA folds upon transcription into a hairpin structure comprising one or more double stranded regions separated by a loop. These RNA molecules act according to an RNA silencing or RNAi mechanism to suppress expression of viral genomic targets of at least two viruses. Alternatively, a nucleic acid guided nuclease and guide RNAs are expressed from the nucleic acid construct leading to cleavage of the viral genomic targets by the nuclease, which is directed to the target by the crRNAs. The resulting Beta plant exhibits resistance against the at least two targeted viruses.

For the transformation step, methods based on biological approaches, like Agrobacterium transformation or viral vector mediated plant transformation, or methods based on physical delivery methods, like particle bombardment or microinjection, can be used for importing the construct or set of constructs into the cell. Currently, there are a variety of plant transformation methods known to the skilled person in the field of plant biotechnology. In a preferred embodiment, the transformation is an Agrobacterium mediated transformation as described by Kishchenko et al., Production of transgenetic sugarbeet (Beta vulgaris L.) plants resistant to phosphinothricin (2005) Cell Biology International, 29(1): 15-19.

The transformed cell is then cultivated under conditions allowing regeneration of a transgenic Beta plant, which then can be tested for the desired resistance(s). Preferably, regeneration of transgenic sugar beet plants after transformation is essentially carried out as described in Kishchenko et al., Production of transgenetic sugarbeet (Beta vulgaris L.) plants resistant to phosphinothricin (2005) Cell Biology International, 29(1): 15-19.

In one embodiment of the method described above, in step (i) a first Beta plant cell is transformed with a nucleic acid construct or a set of nucleic acid constructs comprising sense and/or antisense sequences targeting a genomic sequence of each of two, three, four or five viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV) or orthologous sequences thereof and a second Beta plant cell is transformed with a nucleic acid construct or a set of nucleic acid constructs comprising sense and/or antisense sequences targeting a genomic sequence of each of two, three, four or five of the viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV) or orthologous sequences thereof, and

in step (ii) a first Beta plant is regenerated from the transformed first Beta plant cell and a second Beta plant is regenerated from the transformed second Beta plant cell, and

in step (iii) a first Beta plant is selected and/or obtained, which is resistant to two, three, four or five of the viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV) Beet Western Yellowing Virus (BWYV) and Beet Chlorosis Virus (BChV) and a second Beta plant is selected and/or obtained, which is resistant to two, three, four or five of the viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV), and

the method comprises a step (iv) of crossing the first and the second plant selected and/or obtained in step (iii) to generate an F1 generation of the plants and, optionally,

a step (v) of obtaining and/or selecting a plant, which is resistant to at least four, five, six or all of the viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV).

Preferably, in step (i), the second Beta plant cell is transformed with a nucleic acid construct or a set of nucleic acid constructs comprising sense and/or antisense sequences targeting genomic sequences of viruses, which were not selected for transformation of the first Beta plant cell.

The selection in step (ii) can be done genotypically (by checking for the presence or absence of a given sequence, i.e. in the context of the present invention preferably for the presence of the nucleic acid construct or a set of nucleic acid constructs as described above). Methods for genotypical as well as phenotypical selection for Beta plants showing resistance are known to the person of skill in the art. Preferably, a selection as in step (ii) is done one the basis of resistance assays (phenotypic evaluation of symptoms and virus titer), but also by genotypic characterization.

In this case, crossing of the two plants in step (iv) provides an F1 generation, which comprises plants resistant to a combination of the viruses, which the parent plants were resistant to, preferably resistant to all of the viruses recited above.

Methods of crossing plants of the genus Beta as well as methods for selection of plants of the genus Beta that exhibit a certain characteristic (such as, for example, the resistance to at least two the viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV)) are known to the person of skill in the art.

The transgenic plant resulting from the method above shows resistance to at least two viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV).

In a further aspect, the present invention relates to the use of a nucleic acid construct or a set of nucleic acid constructs according to any of the embodiments described above to confer resistance to at least two viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV) to a Beta plant.

The present invention is further described with reference to the following non-limiting examples as well as the attached sequence listings and figures.

### Example 1: Analysis of Viral Genomes and Selection of Viral Target Sequences for an RNAi Approach

To abolish viral replication, it is essential to identify a target region within the viral genome that is suitable for this approach. Sequences for the economically most relevant sugar beet viruses (BCTV, BSCTV, BYV, BMYV and BNYVV) were selected from Genbank. Different isolates of each virus were compared and analyzed for suitable regions with a size of 400-500 bp in order to engineer RNAi constructs. For BCTV, BSCTV and BMYV preferably overlapping ORFs were selected as RNAi target regions. For those viruses where no overlapping ORF exist other regions were selected. In FIG. 1 the location of the targets in the respective genomes is demonstrated. For the DNA virus BCTV/BSCTV genome, the targets V2/MP/CP; C2/C3 and C4/Rep were selected (FIG. 1A). For BMYV/BWYV/BChV genome, the target P0/P1 and P3/P4 were selected (FIG. 1B); For BYV genome, the targets CDS5, CDS6 and CDS7 were selected (FIG. 1C). For BNYVV genome, the target CP was selected (FIG. 1D). The selected regions were used for the design of RNAi constructs. First, for each hairpin construct only one sense and one antisense region, respectively, was used. For these constructs transgenic Nicotiana benthamiana and/or transgenic sugar beet lines were produced.

### Example 2: Generation of Transgenic Sugar Beet Lines

Sugar beets were transformed by using Agrobacterium mediated transformation based on Kishchenko et al., Production of transgenetic sugarbeet (Beta vulgaris L.) plants resistant to phosphinothricin (2005) Cell Biology International, 29(1): 15-19. Micropropagated shoots of the genotype 5706 were used as starting material. Shoots were multiplied in MS salts supplemented with 30 g/l sucrose and 0.25 mg/l benzyladenine (BAP). To induce friable callus, leaf explants were incubated in MS salts including 15 g/l sucrose and 2 mg/l BAP at around 30° C. for several weeks and friable calli were harvested. Agrobacterium AGL-1 harbouring the RNAi constructs of interest (see Example 1) was grown in suitable medium supplemented with the appropriate antibiotics. Calli were inoculated with Agrobacterium suspension. The co-culture of the callus tissue and the Agrobacterium was done in medium containing 440 mg/l CaCl₂x2H₂O, 170 mg/l KH₂PO₄, 1900 mg/l KNO₃, 370 mg/l MgSO₄, 1650 mg/l NH₄NO₃, 2 mg/l BAP, 40 mg/l Acetosyringone, 20 g/l sucrose and 2 g/l glucose for at least 2 days. Calli were subcultured to MS salts supplemented with 30 g/l sucrose, 1 mg/l GA3, 1 mg/l TDZ and 500 mg/l Timentin and incubated in the dark, for 1 week. For the selection of transgenic cells, calli were transferred to the described medium supplemented with 2.5 g/L D-mannose and incubated in the light for several weeks. Transgenic calli were selected and subcultured for several times in the same medium and conditions. Regenerating shoots were isolated and propagated in MS salts including 30 g/l sucrose, 0.25 mg/l BAP and 2.5 g/L D-Mannose.

Leaf explants were isolated from the green growing shoots for DNA extraction and PCR analysis, in order to confirm the putative transgenic lines.

Selected shoots were rooted in MS salts supplemented with 0.5 mg/l IBA, 100 mg/l cefotaxime and transferred to the green house for seed production.

### Example 3: Evaluation of Different Viral Target Sequences

For the evaluation of the different RNAi constructs, a stepwise approach was used. Some constructs were pretested in the model organism Nicotiana benthamiana (stable transgenic lines) and based on the results, the most effective constructs were transformed in sugar beet. Some constructs were directly transformed in sugar beet. In sugar beet, there were different effects on virus resistance observed.

Resistance tests: The independent transgenic lines were grown and propagated in in-vitro culture in media containing MS salts, 30 g/l sucrose, 250 mg/l Timentin and 0.25 mg/l BAP. For the resistance tests 35 shoots of each line were transferred to rooting media containing MS salts, 30 g/l sucrose and 6.25 mg/l NAA. After 4 weeks roots were formed and the plants were transferred to the green house and potted in soil.

BCTV and BSCTV infection: Two weeks after transfer to the soil, leaves of the sugar beet shoots were vacuum infiltrated with Agrobacteria GV3101 containing an infectious full-length clone of BCTV or BSCTV. For the cultivation of Agrobacteria a fresh cryo culture was used to inoculate a plate with LB medium supplemented with the appropriate antibiotics. After 2-3 days at 28° C. a 50 ml preculture was inoculated from this plate and grown overnight at 28° C. and 200 rpm. From the preculture 400 ml cultures for the infection experiment were inoculated and incubate for approx. 22h at 28° C. and 200 rpm.

For the vacuum infiltration the bacteria were centrifuged at 4400 rpm for 15 minutes. The supernatant was discarded, and the bacteria dissolved in 4-5 ml infiltration medium containing H₂O, 10 mM MES, 10 mM MgCl₂ and 200 μM acetosyringone. The OD600 was measured and adjusted to 1.5 with infiltration medium and incubated for 4 hours at room temperature. Before vacuum infiltration 0.03% Silvett Gold was added to the bacteria suspension and the plants were removed from soil. The infiltration was done in a desiccator connected to a vacuum pump. Leaves were immersed in the bacteria solution and vacuum was applied for 1 minute. After the infiltration procedure the plants were potted and covered with a hood for 1 day.

Virus content was analyzed 4 weeks past infection. Leaf material was used for DNA extraction and subsequent quantitative PCR with BCTV and BSCTV specific Taqman probes.

BMYV (BWYV) infection: A similar protocol as described for the BCTV and BSCTV infection was used. Shoots were vacuum infiltrated with Agrobacteria GV3101 or AGL1 containing an infectious full-length clone of BMYV (binary plasmid pGREEN containing the BMYV genome) or BWYV. The infiltration media contained MS salts, 20 g/l sucrose and 200 μM acetosyringone. Virus content of leaves was analyzed 5 weeks past infection by ELISA. Leaf material from each plant was sampled and used for quantification of the virus content with a standard double antibody sandwich assay (DAS-ELISA) using TuYV antibody (Loewe Biochemica GmbH). We used a dilution of 1:200 and a test volume of 200 μl/well. A details protocol is published at https://www.loewe-info.com/.

BYV infection: Two weeks after transfer to the soil, virus loaded aphids were placed on the leaves sugar beet shoots. Three days after aphid transfer the plants were treated with an insecticide to remove the aphids. Leaf samples were taken six weeks past infection and virus content was determined by DAS-ELISA using BYV antibody (Loewe Biochemica GmbH). We used a dilution of 1:200 and a test volume of 200 μl/well. A detailed protocol is published at https://www.loewe-info.com/.

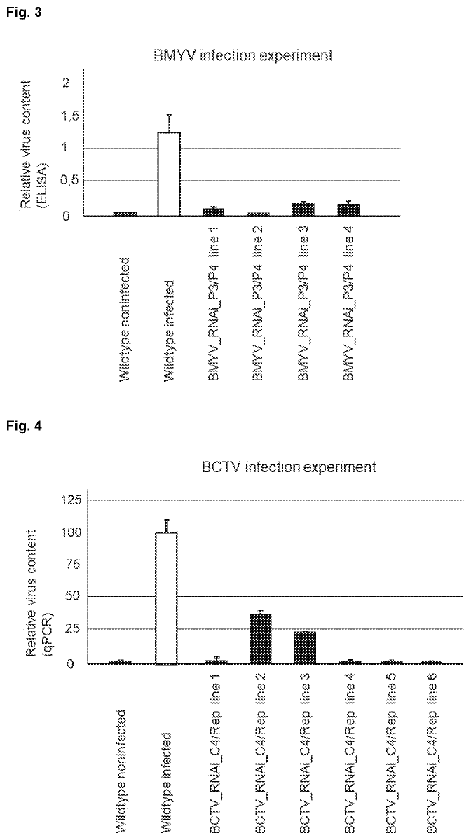

BNYVV infection: The transgenic lines were transferred to soil containing BNYVV and the vector Polymyxa betae. 10 weeks after this transfer the roots were washed, the virus content was determined by DAS ELISA using polyclonal antiserum for BNYVV (Loewe Biochemica GmbH). The effect on resistance was classified as effective (very low virus titer and very weak or no symptoms), less effective (significantly reduced virus titer compared to nontransgenic control plants and weak symptoms). A summary of tested regions and their effect on resistance is shown in Table 2. Examples for effective constructs in respect to virus resistance are shown in FIGS. 2 to 4. For BYV resistance constructs containing the viral region BYV_CDS7 as antisense and sense region were most effective. Results of a representative BYV infection experiment are shown in FIG. 2. For BMYV resistance constructs containing the viral region BMYV_P3/P4 as antisense and sense region were most effective. Results of a representative BMYV infection experiment are shown in FIG. 3. For BCTV resistance constructs containing the viral region BCTV_C4/Rep as antisense and sense region were most effective. Results of a representative BCTV infection experiment are shown in FIG. 4.

TABLE 2
Overview of different tested constructs.

| | N. | B. | Effect on |
|---|---|---|---|
| | benthamiana | vulgaris | resistance |
| BYV_RNAi_CDS5 | X | X | less effective |
| BYV_RNAi_CDS6 | X | X | less effective |
| BYV_RNAi_CDS7 | X | X | effective |
| BYV_RNAi_CDS5/6/7 | X | X | effective |
| BMYV_RNAi_P0/P1 | | X | effective |
| BMYV_RNAi_P3/P4 | | X | effective |
| BMYV_RNAi3 | | X | less effective |
| BMYV_RNAi4 | | X | less effective |
| BMYV_RNAi5 | | X | less effective |
| BCTV_RNAi_C2/C3 | | X | less effective |
| BCTV_RNAi_C4/Rep | | X | effective |
| BCTV_RNAi_V2/MP/CP | | X | less effective |
| BCTV_RNAi_C2/C3/C4/Rep | X | — | effective |
| BCTV_RNAi_C2/C3/C4/Rep/ | | X | effective |
| V2/MP/CP | | | |

For these constructs stable N. benthamiana and/or sugar beet transgenic lines were tested for their effect on virus resistance. The effect on resistance was classified as effective (very low virus titer and very weak or no symptoms), less effective (significantly reduced virus titer compared to nontransgenic control plants and weak symptoms).

### Example 4: Design of Combination Construct for Combined Multiple Virus Resistance

The most effective constructs identified in the previous Examples were combined in different designs using different promoters (Cauliflower mosaic virus d35S promoter, Ubiquitin10 promoter from Arabidopsis, Ubiquitin4 promoter from Parsley, Petroselinum crispum), number of promoters (one, two and three), introns and terminators. An overview of the different designs is shown in FIG. 5. Nine different constructs (MVR004-MVR012) were cloned and transformed into sugar beet as described in Example 2. The schematic structures of the constructs are shown in FIG. 6-14. For selection of the transgenic events phosphomannose-isomerase (PMI) was used as selection system. The presence of the expression cassette was confirmed with PCR analysis. Additionally, the copy number was analyzed using quantitative PCR and only single copy lines and lines with two copies were selected for further analysis and resistance assays.

The resistance assays were performed as described in Example 3.

### Example 5: Expression Analysis and sRNA Quantification

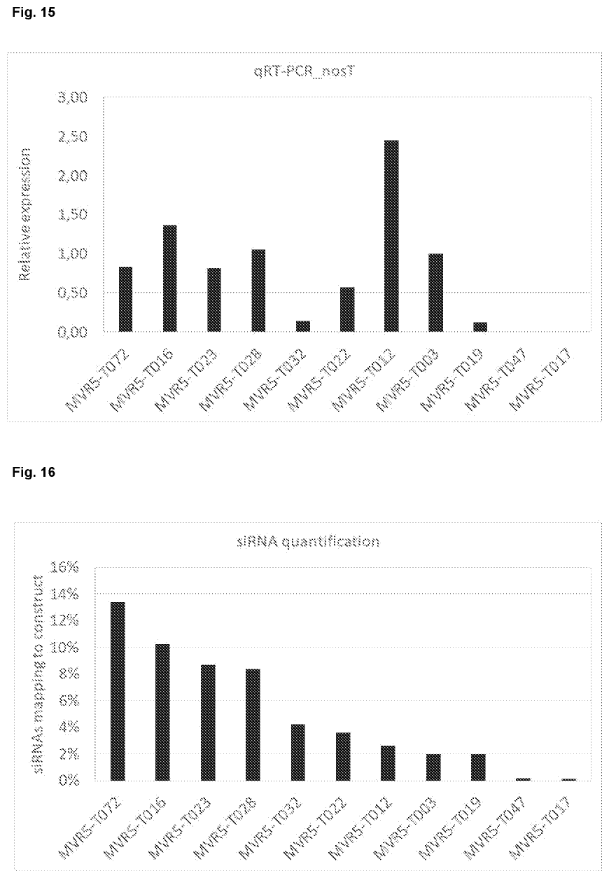

The RNAi approach for virus resistance is based on the plant post-transcriptional gene silencing (PTGS) to degrade viral RNAs. Expression of the inverted-repeat transgene cassette is an essential prerequisite for the formation of an dsRNA hairpin molecule to induce the plant RNAi machinery. Therefore, the transcript amount was analyzed using a quantitative real time PCR. Further, a SYBR assay (Platinum SYBR Green qPCR SuperMix-UDG″ Kit from Invitrogen) was used to detect the transgene transcript using oligonucleotides binding to the nos terminator (NosT_for01 and NosT_rev01). One line was selected, the transcript amount set to 1 and the transcript amounts of the other lines compared to this line. There were significant differences detectable and used for prioritization of the transgenic lines. A representative example of qRT-PCR results of MVR005 transgenic lines is shown in FIG. 15.

It is assumed that a transgenic RNAi line needs a significant amount of small RNA molecules complementary to the viral genome to exhibit strong resistance. Therefore, the siRNA amounts in transgenic lines were quantified. RNA of plant leaf material was extracted using the Direct-zol RNA MiniPrep Kit from Zymo Research. MiRNA libraries were prepared by Eurofins Genomics using the QIAseq miRNA protocol from Qiagen. Libraries were pooled and miRNAs were sequenced in one full flow cell on Illumina HiSeq 2500 using the 1×75 bp read module. A representative example of a siRNA quantification of MVR005 transgenic lines is shown in FIG. 16.

### Example 6: Seed Production, Hybrid Seed Production and Field Testing

Resistant transgenic sugar beet lines will be used for seed production. The shoots will be propagated and rooted as described in Example 3 and transferred to soil. Since sugar beet is a biennial plant it requires for bolting and seed production a prolonged period of cold. 4 weeks after potting from in-vitro culture to soils plants will be transferred for 1 week to acclimation conditions with 12° C., followed by a vernalization period of 12 weeks at 4° C. Before potting in 5 l pots and transfer to the greenhouse the plants will be adapted for additional 2 weeks at 12° C. For the bolting and flowering periods the plants will be covered with a hood until seeds were produced. The seeds will be germinated and using quantitative PCR the homozygous plants will be identified, vernalized as described and planted in field partitions as pollinators for male sterile lines for hybrid seed production. The harvested seeds will be used for field testing.

The resistance tests in the field may be performed using the natural virus vector. For BCTV and BSCTV virus carrying beet leaf hoppers may be used, for BMYV and BYV virus carrying aphids may be used. BNYVV resistance will be tested in regions with virus pressure in the soil.

### SEQUENCE LIST

SEQ ID NO: 1 CDS7_sense sequence from BYV

SEQ ID NO: 2 P3/P4 sense sequence from BMYV

SEQ ID NO: 3 C4/Rep sense sequence from BCTV

SEQ ID NO: 4 C4/Rep sense sequence from BSCTV

SEQ ID NO: 5 CP sense sequence from BNYVV

1. A nucleic acid construct or a set of nucleic acid constructs conferring resistance to a Beta plant to two or more of the viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV), wherein the nucleic acid construct or the set of nucleic acid constructs comprises or encodes at least one sense and/or antisense sequence targeting a genomic sequence of each of at least two viruses, or more than two viruses, selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV) Beet Western Yellowing Virus (BWYV) and Beet Chlorosis Virus (BChV) or orthologue sequences thereof.

2. The nucleic acid construct or set of nucleic acid constructs according to claim 1, wherein the nucleic acid construct or the set of nucleic acid constructs comprises or encodes at least one sense and/or antisense sequence targeting a genomic sequence of each of at least three, at least four, at least five, at least six or all of the viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV), Beet Chlorosis Virus (BChV) or orthologue sequences thereof.

3. The nucleic acid construct or set of nucleic acid constructs according to claim 1, wherein the nucleic acid construct or the set of nucleic acid constructs comprises or encodes sense and/or antisense sequences targeting a genomic sequence of Beet Curly Top Virus (BCTV) and Beet Severe Curly Top Virus (BSCTV) or orthologue sequences thereof, or wherein the nucleic acid construct or the set of nucleic acid constructs comprises or encodes sense and/or antisense sequences targeting a genomic sequence of Beet Curly Top Virus (BCTV), Beet Severe Curly Top Virus (BSCTV) and Beet Necrotic Yellow Vein Virus (BNYVV) or orthologue sequences thereof, or wherein the nucleic acid construct or the set of nucleic acid constructs comprises or encodes sense and/or antisense sequences targeting a genomic sequence of Beet Curly Top Virus (BCTV), Beet Severe Curly Top Virus (BSCTV), Beet Necrotic Yellow Vein Virus (BNYVV), Beet Yellows Virus (BYV) and Beet Mild Yellowing Virus (BMYV) or orthologue sequences thereof or the nucleic acid construct or the set of nucleic acid constructs comprises or encodes sense and/or antisense sequences targeting a genomic sequence of Beet Curly Top Virus (BCTV), Beet Severe Curly Top Virus (BSCTV), Beet Necrotic Yellow Vein Virus (BNYVV), Beet Yellows Virus (BYV) and Beet Western Yellowing Virus (BWYV) or orthologue sequences thereof.

4. The nucleic acid construct or set of nucleic acid constructs according to claim 1, wherein the sense and/or antisense sequence(s) each have a length of at least 20 nt, at least 25 nt, at least 30 nt, at least 40 nt, at least 50 nt, at least 60 nt, at least 70 nt, at least 80 nt, at least 90 nt, at least 100 nt, at least 110 nt, at least 120 nt, at least 130 nt, at least 140 nt, at least 150 nt, at least 160 nt, at least 170 nt, at least 180 nt, at least 190 nt, at least 200 nt, at least 220 nt, at least 250 nt, at least 270 nt or at least 300 nt and/or wherein the sense and/or antisense sequence(s) each have a length of 4000 nt or less, 3500 nt or less, 3000 nt or less, 2000 nt or less or 1000 nt or less.

5. The nucleic acid construct or set of nucleic acid constructs according to claim 1, wherein the sense and/or antisense sequence(s) each have a length of 50 nt to 2000 nt, preferably 100 nt to 1000 nt, more preferably 300 nt to 600 nt.

6. The nucleic acid construct or set of nucleic acid constructs according claim 1, wherein the nucleic acid construct or the set of nucleic acid constructs comprises or encodes at least two corresponding sense and antisense sequences, wherein the sense sequences are selected from the sequences of SEQ ID NO: 1, SEQ ID NO: 2, SEQ ID NO: 3, SEQ ID NO: 4 and SEQ ID NO: 5 or fragments thereof or sequences having a sequence identity of at least 80%, at least 85%, at least 90%, at least 95% to the sequences of SEQ ID NO: 1, SEQ ID NO: 2, SEQ ID NO: 3, SEQ ID NO: 4 and SEQ ID NO: 5 or fragments thereof.

7. The nucleic acid construct or set of nucleic acid constructs according to claim 1, wherein the nucleic acid construct or the set of nucleic acid constructs comprises or encodes at least the sequences of SEQ ID NO: 3 and SEQ ID NO: 4 or fragments thereof or sequences having a sequence identity of at least 80%, at least 85%, at least 90%, at least 95% to the sequences of SEQ ID NO: 3 and SEQ ID NO: 4 or fragments thereof and the corresponding antisense sequences, or wherein the nucleic acid construct or the set of nucleic acid constructs comprises or encodes at least the sequences of SEQ ID NO: 3, SEQ ID NO: 4 and SEQ ID NO: 5 or fragments thereof or sequences having a sequence identity of at least 80%, at least 85%, at least 90%, at least 95% to the sequences of SEQ ID NO: 3, SEQ ID NO: 4 and SEQ ID NO: 5 or fragments thereof and the corresponding antisense sequences, or wherein the nucleic acid construct or the set of nucleic acid constructs comprises or encodes at least the sequences of SEQ ID NO: 1, SEQ ID NO: 2, SEQ ID NO: 3, SEQ ID NO: 4 and SEQ ID NO: 5 or fragments thereof or sequences having a sequence identity of at least 80%, at least 85%, at least 90%, at least 95% to the sequences of SEQ ID NO: 1, SEQ ID NO: 2, SEQ ID NO: 3, SEQ ID NO: 4 and SEQ ID NO: 5 or fragments thereof and the corresponding antisense sequences.

8. The nucleic acid construct or set of nucleic acid constructs according to claim 1, wherein the nucleic acid construct or the set of nucleic acid constructs comprises or encodes the corresponding sense and antisense sequences arranged as follows:
(a) the sense sequences targeting all of the seven viruses recited in claim 1, six of the viruses recited in claim 1, five of the viruses recited in claim 1, four of the viruses recited in claim 1, three of the viruses recited in claim 1 or two of the viruses recited in claim 1 are arranged in contiguous order and the corresponding antisense sequences are arranged in the reverse contiguous order, or
(b) the sense sequences targeting five and two of the viruses recited in claim 1 or four and three of the viruses recited in claim 1 are arranged in contiguous order, respectively, and the corresponding antisense sequences are arranged in the reverse contiguous order, or
(c) the sense sequences targeting five of the viruses recited in claim 1 are arranged in contiguous order and the corresponding antisense sequences are arranged in the reverse contiguous order while the sense sequences targeting the remaining two viruses recited in claim 1 and the corresponding antisense sequences are not arranged in contiguous order, or the sense sequences targeting four and two of the viruses recited in claim 1 are arranged in contiguous order, respectively, and the corresponding antisense sequences are arranged in the reverse contiguous order, or the sense sequences targeting three and three of the viruses recited in claim 1 are arranged in contiguous order, respectively, and the corresponding antisense sequences are arranged in the reverse contiguous order, or the sense sequences targeting three, two and two of the viruses recited in claim 1 are arranged in contiguous order, respectively, and the corresponding antisense sequences are arranged in the reverse contiguous order, or
(d) the sense sequences targeting two, two and two of the viruses recited in claim 1 are arranged in contiguous order, respectively, and the corresponding antisense sequences are arranged in the reverse contiguous order, or the sense sequences targeting three and two of the viruses recited in claim 1 are arranged in contiguous order, respectively, and the corresponding antisense sequences are arranged in the reverse contiguous order while the sense sequences targeting the remaining two viruses recited in claim 1 and the corresponding antisense sequences are not arranged in contiguous order, or the sense sequences targeting four of the viruses recited in claim 1 are arranged in contiguous order and the corresponding antisense sequences are arranged in the reverse contiguous order while the sense sequences targeting the remaining three viruses recited in claim 1 and the corresponding antisense sequences are not arranged in contiguous order, or
(e) the sense sequences targeting two and two of the viruses recited in claim 1 are arranged in contiguous order, respectively, and the corresponding antisense sequences are arranged in the reverse contiguous order while the sense sequences targeting the remaining three viruses recited in claim 1 and the corresponding antisense sequences are not arranged in contiguous order, or the sense sequences targeting three of the viruses recited in claim 1 are arranged in contiguous order and the corresponding antisense sequences are arranged in the reverse contiguous order while the sense sequences targeting the remaining four viruses recited in claim 1 and the corresponding antisense sequences are not arranged in contiguous order, or
(f) the sense sequences targeting two of the viruses recited in claim 1 are arranged in contiguous order and the corresponding antisense sequences are arranged in the reverse contiguous order while the sense sequences targeting the remaining five viruses recited in claim 1 and the corresponding antisense sequences are not arranged in contiguous order, or
(g) none of the sense sequences targeting the seven viruses recited in claim 1 or the corresponding antisense sequences are arranged in contiguous order.

9. The nucleic acid construct or set of nucleic acid constructs according to claim 8, wherein the sense sequences are selected from the sequences of SEQ ID NO: 1, SEQ ID NO: 2, SEQ ID NO: 3, SEQ ID NO: 4 and SEQ ID NO: 5 or fragments thereof or sequences having a sequence identity of at least 80%, at least 85%, at least 90%, at least 95% to the sequences of SEQ ID NO: 1, SEQ ID NO: 2, SEQ ID NO: 3, SEQ ID NO: 4 and SEQ ID NO: 5 or fragments thereof.

10. An RNA molecule or a set of RNA molecules, which is/are formed upon transcription of a nucleic acid construct or set of nucleic acid constructs according to claim 1.

11. A vector or set of vectors comprising a nucleic acid construct or set of nucleic acid constructs according to claim 1.

12. A virus resistant transgenic Beta plant, cell of a Beta plant, part of a Beta plant or seed of a Beta plant comprising a nucleic acid construct or set of nucleic acid constructs according to claim 1, or comprising an RNA molecule or set of RNA molecules formed upon transcription of a nucleic acid construct or set of nucleic acid constructs according to claim 1, or comprising a vector or set of vectors comprising a nucleic acid construct or set of nucleic acid constructs according to claim 1.

13. A method of conferring resistance to a Beta plant to at least two viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV) comprising the steps:
(i) transforming at least one Beta plant cell with a nucleic acid construct or set of nucleic acid constructs according to claim 1 or a vector or set of vectors comprising a nucleic acid construct or set of nucleic acid constructs according to claim 1, and
(ii) regenerating a Beta plant from the transformed cell of step (i),
(iii) optionally, selecting and/or obtaining a Beta plant, which is resistant to at least two viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV) Beet Western Yellowing Virus (BWYV) and Beet Chlorosis Virus (BChV).

14. The method according to claim 13, wherein
in step (i) a first Beta plant cell is transformed with a nucleic acid construct or a set of nucleic acid constructs comprising sense and/or antisense sequences targeting a genomic sequence of each of two, three, four or five viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV) or orthologous sequences thereof and a second Beta plant cell is transformed with a nucleic acid construct or a set of nucleic acid constructs comprising sense and/or antisense sequences targeting a genomic sequence of each of two, three, four or five of the viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV) or orthologous sequences thereof, and
in step (ii) a first Beta plant is regenerated from the transformed first Beta plant cell and a second Beta plant is regenerated from the transformed second Beta plant cell, and
in step (iii) a first Beta plant is selected and/or obtained, which is resistant to two, three, four or five of the viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV) Beet Western Yellowing Virus (BWYV) and Beet Chlorosis Virus (BChV) and a second Beta plant is selected and/or obtained, which is resistant to two, three, four or five of the viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV), and
the method comprises a step (iv) of crossing the first and the second plant selected and/or obtained in step (iii) to generate an F1 generation of the plants and, optionally,
a step (v) of obtaining and/or selecting a plant, which is resistant to at least four, five, six or all of the viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV).

15. A use of a nucleic acid construct or a set of nucleic acid constructs according to claim 1 to confer resistance to at least two viruses selected from the group consisting of Beet Curly Top Virus (BCTV), Beet Severe Curly Top Virus (BSCTV), Beet Necrotic Yellow Vein Virus (BNYVV), Beet Yellows Virus (BYV), Beet Mild Yellowing Virus (BMYV), Beet Western Yellowing Virus (BWYV) and Beet Chlorosis Virus (BChV) to a Beta plant.
